SEQUENCE LISTING (14 sequences)

SEQ ID NO: 1 - protein, 323 aa, Homo sapiens
MSDRPTARRWGKCGPLCTRENIMVAFKGVWTQAFWKAVTAEFLAMLIFVLLSLGSTINWGGTEKPLPVDMVLISLCFGLSIATMVQCFGHISGGHINPAVTVAMVCTRKISIAKSVFYIAAQCLGAIIGAGILYLVTPPSVVGGLGVTMVHGNLTAGHGLLVELIITFQLVFTIFASCDSKRTDVTGSIALAIGFSVAIGHLFAINYTGASMNPARSFGPAVIMGNWENHWIYWVGPIIGAVLAGGLYEYVFCPDVEFKRRFKEAFSKAAQQTKGSYMEVEDNRSQVETDDLILKPGVVHVIDVDRGEEKKGKDQSGEVLSSV

SEQ ID NO: 2 - DNA, 5216 bp, Homo sapiens
gactcccagcacacagggagctgcggggcaggcaatgagagctgcactctggctggggaaggcatgagtgacagacccacagcaaggcggtggggtaagtgtggacctttgtgtaccagagagaacatcatggtggctttcaaaggggtctggactcaagctttctggaaagcagtcacagcggaatttctggccatgcttatttttgttctcctcagcctgggatccaccatcaactggggtggaacagaaaagcctttaccggtcgacatggttctcatctccctttgctttggactcagcattgcaaccatggtgcagtgctttggccatatcagcggtggccacatcaaccctgcagtgactgtggccatggtgtgcaccaggaagatcagcatcgccaagtctgtcttctacatcgcagcccagtgcctgggggccatcattggagcaggaatcctctatctggtcacacctcccagtgtggtgggaggcctgggagtcaccatggttcatggaaatcttaccgctggtcatggtctcctggttgagttgataatcacatttcaattggtgtttactatctttgccagctgtgattccaaacggactgatgtcactggctcaatagctttagcaattggattttctgttgcaattggacatttatttgcaatcaattatactggtgccagcatgaatcccgcccgatcctttggacctgcagttatcatgggaaattgggaaaaccattggatatattgggttgggcccatcataggagctgtcctcgctggtggcctttatgagtatgtcttctgtccagatgttgaattcaaacgtcgttttaaagaagccttcagcaaagctgcccagcaaacaaaaggaagctacatggaggtggaggacaacaggagtcaggtagagacggatgacctgattctaaaacctggagtggtgcatgtgattgacgttgaccggggagaggagaagaaggggaaagaccaatctggagaggtattgtcttcagtatgactagaagatcgcactgaaagcagacaagactccttagaactgtcctcagatttccttccacccattaaggaaacagatttgttataaattagaaatgtgcaggtttgttgtttcatgtcatattactcagtctaaacaataaatatttcataatttacaaaggaggaacggaagaaacctattgtgaattccaaatctaaaaaaagaaatatttttaaaatgttcttaagcaaatatatacctattttatctagttacctttcattaacaaccaattttaaccgtgtgtcaagatttggttaagtcttgcctgacagaactcaaagacacgtctatcagcttattccttctctactggaatattggtatagtcaattcttatttgaatatttattctattaaactgagtttaacaatggcaaaatacagtatgtcacagtcatgcacattcaagagagaaaatataacaagttcttttatgagcaatcccttatgcatagactaccttggcaaaagagcattagcaagtgtcactgctcatcagttacttccttccatttatatcacaaatacccaagtttcaattctaacttcatttcatggtatttcttcctcctcaatgcccaaggtaatgtgggactaaagcccagaaatttgaaaagaatattcagaaatccttcccaaatcataagggcacctattgagattcaagacaagcagactcgtaaaatcttgtagaggcagaggcaaagttatcatcatacaaaaatcacaaacaaaaaggagatctgtattcgggtatcaaacggttgatctgttttcagtgcacaccctcaaatgcaccacaccagttttacgatctaagcttttaacccctttaccactttgcttatttttaaaaaatttattggcaaaactggggattttgtttgtaactttggttatctatatttaaacattagctgagacatatttttgataacaagaacttagctagttgagtcctggctttttgttgattattaagctgtgttcactctagaaggtgtaaaggccctgtcccaatctctgctctctcacttctcgtgacatttgttgataatctttactgcaagtgtaatgatatttgagtgaattaaaatgcatgacagacaaggtacaacatggttgcagaataaaagggtgaggacatcagatcactctgtattcaaagcttactagacgaacaatctttcatttctggatatattgtagctaacccaacacatttttaaactatttttgttactgactcacttacttgtcaaaataattggtttttctgtcctgctttgccttttgtcactagaatgttctccagaaaccccagtttctgtcatcttaactgtctagtctagccgttaacttagacaaagaagtgcaaatgtcaatttcactaaacaaaagctattagaagtaaagcagaaacctatcactgcaaggagtgacttgtaaataactgatgatcaagggctgtcttctctcaagcatggttccaactctgtgtcactttttagcgttaatggcagttgtgtgtctgtggcagtgagataatggaccactattaaacctgattctcttcggtgctaggaaagagtgatgtgtgagattcccagagagtcgtcacaccagtgttatgcctgggagtgccactggcataccgcaggtgcctaaatcccaggcccctttctcagggggcgtggccacaccacacactgcgccagtgacacatagttacttcctggctgggacggcacctatagaaactagggtacaaatctcccaaagatggtttgcaccgtcatttcaaatgtttgcacaatataccacttagaggcttaaaatcagggtggttcagttcaaaaggtaatatcaaccgtcagcaaacatgtcagtttaaaaataagttcggttaacaaggaagtgttaagcagattttttgaatacacttacaatatactggcttataatcatttttattctgccatatggtttatttcatatacactcacttttacactcacttccaattatagcattcaataagttacggttaaagaaaagtgaatggtgtttttgtaatttgttttctagtagctagcagagagcctggcacatagtatgtattcaaaacaagtgtgttaattaataaaaatatataaatctcatttgagatttaagaatataaataattttcttttaatctttattttgctcaatatttaataggaagtatgatttcaaggactactaaaggaaggcaatatacatgtgaaaaccataaaatacattttttgcattctactatttttaggggcatgaaatgaatgaaagctttagcacttttttgcccctacatatgagacaacatattttgtatttcactcaaggctctgccagtaaaaagtaatgaaattgtacctttctaatgacatctatgcagcaggggtctatcgccttgtggatggcaccaaattatccaagtgtattaggagactggggcttttttctttaatcccttcttattaatgaagtgcatagtgctgctcccaggagaccactgctgacagatacacagagaagagatcagagaggaaaaactgggaagacataaatgaattatacccagccatgaaacaatgccaactgtctcttccctaaggaagagtacaagtaccctaaaattgaaaggtggtccctacactgaaaacgcacatagtttgtcaaaagtgtacaaaagggaaagagtcttattttaagctttcaggctttcttaaaaacttggggaccagaatttcaatgtatgtttccattgttgaagataacattttcttcaaagagccttaaccttttgtactggaaggaaatattttctggacttaagtagttgcctaaatttaagattcctacactttatttctgccattgatgcttttcctaaacccttatactatctttttattatctgagccttttcctaatgcagctcataggtgctagctagagctgctgctcagtattgaagactttacaaggagattagaaatctttggaaaacatatgtgatgaaattgagctatatgatttatcagagatctgattccaaagagcacagaatactgttctcagaccatgaaaccagacaacacatgtattggtttaaactcgataatgacaggaaaattccaaactagagcagtaaattcaaatggtaagatgaatcctagaaggcctctgattgcagcatgttgacaccaacctcacgttacgaacaattcacagagaatttgcctttgtggcaactgaagatggaagtctggggggcacagacaaccttatcaaacaatataaaagccaatataaattctcataagcactatagaatttgcaaattcagaacattttatacctaaaagtaattctgtctttcctaaagtgtttttaacatgaaaattagtaggaagatgtggttactatttggaaagtgtaatgtaacaaaactctcttttgttaccacaaattttgtgagtttagtactctacagattgccccataagagcagtagcttttgaaactcataattctctgaaataaatgaaagacatttaattcaaggatcaaaaattgtggccatctttgcaaatgactacctatagcctgtgaaaatacatttcaaaaaatgttatgtgcaatgaacactaaatttaagagcagttacagtgtgactcactcatgtttaaaaaaaatcgaagagctaaaaaatacgtctaatttatgtaacccattggaatgtatttctaggttctcttcaggattaattaaataaacatgcaatttatgaaaacatataaacaattatttatcacttttatgacccaaatcacaataaaattgtcatttaggataaactggggagaatagactgaacatatggttatattcacagttatttattaacttaaatgttattccaacattagagctaatgttaaaaagatttaaactgtaacgtctaatatttggaataatatattaaagtattagcactgtggttgattttcttgaattatgttgcatcttgtactactaagcttgtgaaaataaacatttggatgttttaaaaggtaaaaaaaaaaaaaaaa

SEQ ID NO: 3 - protein, 301 aa, Homo sapiens
MVAFKGVWTQAFWKAVTAEFLAMLIFVLLSLGSTINWGGTEKPLPVDMVLISLCFGLSIATMVQCFGHISGGHINPAVTVAMVCTRKISIAKSVFYIAAQCLGAIIGAGILYLVTPPSVVGGLGVTMVHGNLTAGHGLLVELIITFQLVFTIFASCDSKRTDVTGSIALAIGFSVAIGHLFAINYTGASMNPARSFGPAVIMGNWENHWIYWVGPIIGAVLAGGLYEYVFCPDVEFKRRFKEAFSKAAQQTKGSYMEVEDNRSQVETDDLILKPGVVHVIDVDRGEEKKGKDQSGEVLSSV

SEQ ID NO: 4 - DNA, 5136 bp, Homo sapiens
tgtttctcttttcagtaagtgtggacctttgtgtaccagagagaacatcatggtggctttcaaaggggtctggactcaagctttctggaaagcagtcacagcggaatttctggccatgcttatttttgttctcctcagcctgggatccaccatcaactggggtggaacagaaaagcctttaccggtcgacatggttctcatctccctttgctttggactcagcattgcaaccatggtgcagtgctttggccatatcagcggtggccacatcaaccctgcagtgactgtggccatggtgtgcaccaggaagatcagcatcgccaagtctgtcttctacatcgcagcccagtgcctgggggccatcattggagcaggaatcctctatctggtcacacctcccagtgtggtgggaggcctgggagtcaccatggttcatggaaatcttaccgctggtcatggtctcctggttgagttgataatcacatttcaattggtgtttactatctttgccagctgtgattccaaacggactgatgtcactggctcaatagctttagcaattggattttctgttgcaattggacatttatttgcaatcaattatactggtgccagcatgaatcccgcccgatcctttggacctgcagttatcatgggaaattgggaaaaccattggatatattgggttgggcccatcataggagctgtcctcgctggtggcctttatgagtatgtcttctgtccagatgttgaattcaaacgtcgttttaaagaagccttcagcaaagctgcccagcaaacaaaaggaagctacatggaggtggaggacaacaggagtcaggtagagacggatgacctgattctaaaacctggagtggtgcatgtgattgacgttgaccggggagaggagaagaaggggaaagaccaatctggagaggtattgtcttcagtatgactagaagatcgcactgaaagcagacaagactccttagaactgtcctcagatttccttccacccattaaggaaacagatttgttataaattagaaatgtgcaggtttgttgtttcatgtcatattactcagtctaaacaataaatatttcataatttacaaaggaggaacggaagaaacctattgtgaattccaaatctaaaaaaagaaatatttttaaaatgttcttaagcaaatatatacctattttatctagttacctttcattaacaaccaattttaaccgtgtgtcaagatttggttaagtcttgcctgacagaactcaaagacacgtctatcagcttattccttctctactggaatattggtatagtcaattcttatttgaatatttattctattaaactgagtttaacaatggcaaaatacagtatgtcacagtcatgcacattcaagagagaaaatataacaagttcttttatgagcaatcccttatgcatagactaccttggcaaaagagcattagcaagtgtcactgctcatcagttacttccttccatttatatcacaaatacccaagtttcaattctaacttcatttcatggtatttcttcctcctcaatgcccaaggtaatgtgggactaaagcccagaaatttgaaaagaatattcagaaatccttcccaaatcataagggcacctattgagattcaagacaagcagactcgtaaaatcttgtagaggcagaggcaaagttatcatcatacaaaaatcacaaacaaaaaggagatctgtattcgggtatcaaacggttgatctgttttcagtgcacaccctcaaatgcaccacaccagttttacgatctaagcttttaacccctttaccactttgcttatttttaaaaaatttattggcaaaactggggattttgtttgtaactttggttatctatatttaaacattagctgagacatatttttgataacaagaacttagctagttgagtcctggctttttgttgattattaagctgtgttcactctagaaggtgtaaaggccctgtcccaatctctgctctctcacttctcgtgacatttgttgataatctttactgcaagtgtaatgatatttgagtgaattaaaatgcatgacagacaaggtacaacatggttgcagaataaaagggtgaggacatcagatcactctgtattcaaagcttactagacgaacaatctttcatttctggatatattgtagctaacccaacacatttttaaactatttttgttactgactcacttacttgtcaaaataattggtttttctgtcctgctttgccttttgtcactagaatgttctccagaaaccccagtttctgtcatcttaactgtctagtctagccgttaacttagacaaagaagtgcaaatgtcaatttcactaaacaaaagctattagaagtaaagcagaaacctatcactgcaaggagtgacttgtaaataactgatgatcaagggctgtcttctctcaagcatggttccaactctgtgtcactttttagcgttaatggcagttgtgtgtctgtggcagtgagataatggaccactattaaacctgattctcttcggtgctaggaaagagtgatgtgtgagattcccagagagtcgtcacaccagtgttatgcctgggagtgccactggcataccgcaggtgcctaaatcccaggcccctttctcagggggcgtggccacaccacacactgcgccagtgacacatagttacttcctggctgggacggcacctatagaaactagggtacaaatctcccaaagatggtttgcaccgtcatttcaaatgtttgcacaatataccacttagaggcttaaaatcagggtggttcagttcaaaaggtaatatcaaccgtcagcaaacatgtcagtttaaaaataagttcggttaacaaggaagtgttaagcagattttttgaatacacttacaatatactggcttataatcatttttattctgccatatggtttatttcatatacactcacttttacactcacttccaattatagcattcaataagttacggttaaagaaaagtgaatggtgtttttgtaatttgttttctagtagctagcagagagcctggcacatagtatgtattcaaaacaagtgtgttaattaataaaaatatataaatctcatttgagatttaagaatataaataattttcttttaatctttattttgctcaatatttaataggaagtatgatttcaaggactactaaaggaaggcaatatacatgtgaaaaccataaaatacattttttgcattctactatttttaggggcatgaaatgaatgaaagctttagcacttttttgcccctacatatgagacaacatattttgtatttcactcaaggctctgccagtaaaaagtaatgaaattgtacctttctaatgacatctatgcagcaggggtctatcgccttgtggatggcaccaaattatccaagtgtattaggagactggggcttttttctttaatcccttcttattaatgaagtgcatagtgctgctcccaggagaccactgctgacagatacacagagaagagatcagagaggaaaaactgggaagacataaatgaattatacccagccatgaaacaatgccaactgtctcttccctaaggaagagtacaagtaccctaaaattgaaaggtggtccctacactgaaaacgcacatagtttgtcaaaagtgtacaaaagggaaagagtcttattttaagctttcaggctttcttaaaaacttggggaccagaatttcaatgtatgtttccattgttgaagataacattttcttcaaagagccttaaccttttgtactggaaggaaatattttctggacttaagtagttgcctaaatttaagattcctacactttatttctgccattgatgcttttcctaaacccttatactatctttttattatctgagccttttcctaatgcagctcataggtgctagctagagctgctgctcagtattgaagactttacaaggagattagaaatctttggaaaacatatgtgatgaaattgagctatatgatttatcagagatctgattccaaagagcacagaatactgttctcagaccatgaaaccagacaacacatgtattggtttaaactcgataatgacaggaaaattccaaactagagcagtaaattcaaatggtaagatgaatcctagaaggcctctgattgcagcatgttgacaccaacctcacgttacgaacaattcacagagaatttgcctttgtggcaactgaagatggaagtctggggggcacagacaaccttatcaaacaatataaaagccaatataaattctcataagcactatagaatttgcaaattcagaacattttatacctaaaagtaattctgtctttcctaaagtgtttttaacatgaaaattagtaggaagatgtggttactatttggaaagtgtaatgtaacaaaactctcttttgttaccacaaattttgtgagtttagtactctacagattgccccataagagcagtagcttttgaaactcataattctctgaaataaatgaaagacatttaattcaaggatcaaaaattgtggccatctttgcaaatgactacctatagcctgtgaaaatacatttcaaaaaatgttatgtgcaatgaacactaaatttaagagcagttacagtgtgactcactcatgtttaaaaaaaatcgaagagctaaaaaatacgtctaatttatgtaacccattggaatgtatttctaggttctcttcaggattaattaaataaacatgcaatttatgaaaacatataaacaattatttatcacttttatgacccaaatcacaataaaattgtcatttaggataaactggggagaatagactgaacatatggttatattcacagttatttattaacttaaatgttattccaacattagagctaatgttaaaaagatttaaactgtaacgtctaatatttggaataatatattaaagtattagcactgtggttgattttcttgaattatgttgcatcttgtactactaagcttgtgaaaataaacatttggatgttttaaaaggtaaaaaaaaaaaaaaaa

SEQ ID NO: 5 - protein, 8 aa, Homo sapiens
GTEKPLPV

SEQ ID NO: 6 - protein, 21 aa, Homo sapiens
TPPSVVGGLGVTMVHGNLTAG

SEQ ID NO: 7 - protein, 6 aa, Homo sapiens
GNWENH

SEQ ID NO: 8 - protein, 263 aa, Rattus norvegicus
MWELRSASFWRAIFAEFFATLFYVFFGLGSSLRWAPGPLHVLQVALAFGLALATLVQTVGHISGAHVNPAVTFAFLVGSQMSLLRAFCYIAAQLLGAVAGAAVLYSVTPPAVRGNLALNTLHAGVSVGQATTVEIFLTLQFVLCIFATYDERRNGRMGSVALAVGFSLTLGHLFGMYYTGAGMNPARSFAPAILTRNFSNHWVYWVGPIIGGGLGSLLYDFLLFPRLKSVSERLSILKGARPSDSNGQPEGTGEPVELKTQAL

SEQ ID NO: 9 - protein, 263 aa, Mus musculus
MWELRSASFWRAIFAEFFATLFYVFFGLGASLRWAPGPLHVLQVALAFGLALATLVQTVGHISGAHVNPAVTFAFLVGSQMSLLRAFCYIAAQLLGAVAGAAVLYSVTPPAVRGNLALNTLHAGVSVGQATTVEIFLTLQFVLCIFATYDERRNGRMGSVALAVGFSLTLGHLFGMYYTGAGMNPARSFAPAILTRNFSNHWVYWVGPIIGGGLGSLLYDFLLFPRLKSVSERLSILKGARPSDSNGQPEGTGEPVELKTQAL

SEQ ID NO: 10 - protein, 263 aa, Homo sapiens
MWELRSASFWRAIFAEFFATLFYVFFGLGSSLRWAPGPLHVLQVAMAFGLALATLVQSVGHISGAHVNPAVTFAFLVGSQMSLLRAFCYMAAQLLGAVAGAAVLYSVTPPAVRGNLALNTLHPAVSVGQATTVEIFLTLQFVLCIFATYDERRNGQLGSVALAVGFSLALGHLFGMYYTGAGMNPARSFAPAILTGNFTNHWVYWVGPIIGGGLGSLLYDFLLFPRLKSISERLSVLKGAKPDVSNGQPEVTGEPVELNTQAL

SEQ ID NO: 11 - protein, 323 aa, Rattus norvegicus
MSDGAAARRWGKCGPPCSRESIMVAFKGVWTQAFWKAVTAEFLAMLIFVLLSVGSTINWGGSENPLPVDMVLISLCFGLSIATMVQCFGHISGGHINPAVTVAMVCTRKISIAKSVFYITAQCLGAIIGAGILYLVTPPSVVGGLGVTTVHGNLTAGHGLLVELIITFQLVFTIFASCDSKRTDVTGSVALAIGFSVAIGHLFAINYTGASMNPARSFGPAVIMGNWENHWIYWVGPIIGAVLAGALYEYVFCPDVELKRRLKEAFSKAAQQTKGSYMEVEDNRSQVETEDLILKPGVVHVIDIDRGDEKKGKDSSGEVLSSV

SEQ ID NO: 12 - protein, 323 aa, Mus musculus
MSDRAAARRWGKCGHSCSRESIMVAFKGVWTQAFWKAVSAEFLATLIFVLLGVGSTINWGGSENPLPVDMVLISLCFGLSIATMVQCFGHISGGHINPAVTVAMVCTRKISIAKSVFYIIAQCLGAIIGAGILYLVTPPSVVGGLGVTTVHGNLTAGHGLLVELIITFQLVFTIFASCDSKRTDVTGSIALAIGFSVAIGHLFAINYTGASMNPARSFGPAVIMGNWANHWIYWVGPIMGAVLAGALYEYVFCPDVELKRRLKEAFSKAAQQTKGSYMEVEDNRSQVETEDLILKPGVVHVIDIDRGEEKKGKDSSGEVLSSV

SEQ ID NO: 13 - protein, 323 aa, Homo sapiens; identical to SEQ ID NO: 1

SEQ ID NO: 14 - protein, 255 aa, Cicadella viridis
MAADKSVDNTKKIIGIDDITDTKTIWRCLAAELIGTLLLVLIGTGSCTGVQISEGDVVVRIALTFGFIIATMVQCIGHVSGCHINPAVTCGLLVTGHISILKAIFYIIVQCVGAIAGSAILKVITPAEFRGTLCMTSLAPGVTPPMGFLVEACITFVLILLVQSVCDDRRKNLGNAAPVAVGLAITCCHLAAIKYTGSSMNPARSFGPAVNGDDNWANHWVYWAGPIVGGVVAGITYRALFRARKPEEEASSYDF

(19) United States
(12) Patent Application Publication
(10) Pub. No.: US 2017/0080063 A1
(43) Pub. Date: Mar. 23, 2017

(54) HIGHLY SOLUBLE AQUAPORIN-4 EXTRACELLULAR LOOP C PEPTIDE IMMUNIZATION FOR TREATMENT OF NEUROMYELITIS OPTICA

(71) Applicant: THE JOHNS HOPKINS UNIVERSITY, Baltimore, MD (US)

(72) Inventor: Michael Levy, Phoenix, MD (US)

(21) Appl. No.: 15/312,368

(22) PCT Filed: May 19, 2015
(86) PCT No.: PCT/US2015/031514

§ 371 Date: Nov. 18, 2016

Related U.S. Application Data
(60) Provisional application No. 62/000,356, filed on May 19, 2014.

(51) Int. Cl. A61K 39/00; A61K 49/00; G01N 33/68; A61K 45/06

(52) U.S. Cl. CPC A61K 39/0008; A61K 45/06; A61K 49/0008; G01N 33/6854; A61K 2039/577; G01N 2800/285

(57) ABSTRACT

The present invention provides pharmaceutical compositions for treating neuromyelitis optica (NMO) comprising a therapeutically effective amount of loop C sequence-containing peptide of aquaporin-4 (AQP4) water channel, or a therapeutically effective fragment or variant thereof. The invention also provides methods for treating NMO by administering therapeutically effective amounts of loop C sequence-containing peptide(s) of AQP4, optionally in an immunosuppressive setting, and also provides diagnostics for detection of NMO in a subject, screening methods for identification of NMO-treating therapeutics and NMO model systems.

### CROSS-REFERENCE TO RELATED APPLICATION

The present invention claims priority to, and the benefit under 35 U.S.C. §119(e) of U.S. provisional patent application No. 62/000,356, entitled “Highly Soluble Aquaporin-4 Extracellular Loop C Peptide Immunization for Treatment of Neuromyelitis Optica,” filed May 19, 2014. The entire contents of the aforementioned patent application are incorporated herein by this reference.

### FEDERALLY SPONSORED RESEARCH

This work was supported by the following grant from the National Institutes of Health, Institute of Neurologic Disease and Stroke: NS078555. The Government has certain rights in the invention.

### INCORPORATION BY REFERENCE

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the invention.

### BACKGROUND

Field of the Invention

The invention generally relates to neurological disorders and treatments thereof, and more particularly, to an antigen-based peptide and pharmaceutical compositions comprising same for treating neuromyelitis optica (NMO).

Background of the Invention

Neuromyelitis optica (NMO), also known as Devic's syndrome, is a devastating, life-threatening, ultra-rare neurological disease that leads to severe weakness, paralysis, respiratory failure, loss of bowel and bladder function, blindness and premature death. It can be characterized as a heterogeneous condition consisting of recurrent and simultaneous inflammation and demyelination of the optic nerve (optic neuritis) and the spinal cord (myelitis), which cause characteristic NMO lesions to form on these tissues. Patients with NMO have life-long repeated episodes of autoimmune attack, and most patients experience an unpredictable, relapsing course of disease with cumulative disability, as each attack adds to the neurologic disability. NMO has both a monophasic and relapsing form, but the relapsing form comprises more than 90% of cases. NMO is an orphan disease with an estimated prevalence rate of 0.32-2.5 cases per 100,000 in the population, affecting approximately 4,000 people in the United States.

NMO is considered an autoimmune disorder. The disease is thought to involve an autoimmune reaction against aquaporin-4 (AQP4), an astrocytic water channel protein located on astrocytes of the optic nerves and spinal cord causing the associated inflammation and demyelination processes characteristic of the disorder. However, the exact nature of the autoimmunogenic process is not well understood.

Unfortunately, there is no cure for NMO. Current management focuses primarily on using immunosuppression in an attempt to reduce the frequency and severity of attacks. Medical practitioners generally treat an initial attack of NMO with a combination of a corticosteroid drug (e.g., methylprednisolone) to stop the attack, and an immunosuppressive drug (e.g., azathioprine) for prevention of subsequent attacks. Despite these approaches, many are left with impairment of limbs, reduced mobility, and loss of vision. In particular, approximately 60% of patients with NMO disease have permanent visual loss in at least one eye and 52% have permanent weakness in at least one limb within 5 years of onset of disease. The mortality rate is 25-32% usually due to transverse myelitis in the cervical spinal cord resulting in respiratory failure.

Given the lack of available treatments and the fact that no cure for NMO is yet available, there is a need in the field for new and improved therapies for treating and/or ameliorating this condition. The present invention addresses this need by providing a newly discovered target for treating and/or ameliorating neuromyelitis optica (NMO), as well as compositions and methods for same.

### SUMMARY OF THE INVENTION

The present invention relates, in part, to the identification of a unique aquaporin-4 peptide (loop C sequence, including fragment(s) comprising loop C sequence, e.g., LVTPPSVVGGLGVTMVHGN (SEQ ID NO: 8)) that is able to trigger pathogenic T cell proliferation in AQP4-knockout mice. This surprising and previously unknown observation forms the basis of antigen-based peptide drug design for the treatment and/or amelioration of neuromyelitis optica (NMO). Accordingly, the present invention relates to pharmaceutical compositions for immunizing and/or inducing tolerance in individuals having NMO. In addition, the invention relates to methods of treating and/or immunizing NMO-patients by administering or immunizing the subjects with therapeutically effective amounts of the loop C and/or loop C sequence-containing peptide(s), or therapeutically effective variants and/or fragments thereof. Treatment may optionally be conducted under a setting of immunosuppression with a drug, such as, rituximab, or as a monotherapy. Administration may be by any suitable means, including oral, intravenous, subcutaneous, or intra-arterial. Immunization is also contemplated by way of using nucleic acid molecules encoding the loop C and/or loop C sequence-containing peptide. Thus, the invention also pertains to nucleic acid molecules encoding the loop C and/or loop C sequence-containing peptide, and to formulations and pharmaceutical compositions for administering such molecules such that they are expressed as antigens in the individual. In addition to a therapeutic product(s), the invention relates, at least in part, to diagnostic testing for presence of loop-C activated T cells in a subject (e.g., a human subject) as a marker of NMO disease.

As discussed, neuromyelitis optica (NMO) is a relapsing autoimmune disease primarily targeting the spinal cord and optic nerve leading to paralysis and blindness. NMO is associated with an antibody against the astrocytic water channel, aquaporin (AQP4). While the anti-AQP4 antibody has been shown to have a pathogenic role in exacerbating disease pathogenesis in animal models, the precise role of T cells in NMO remains unknown. However, T cells are readily detectable in active NMO lesions and AQP4-reactive T cells are necessary for the production of high affinity anti-AQP4 IgG1 antibodies.

Recently, NMO patients were found to have T cells reactive against several discrete AQP4 determinants, including the three extracellular domains targeted by the anti-AQP4 antibody. However, it was not understood whether such T cells had a role, if any, in disease pathogenesis.

The inventors sought to generate AQP4-reactive T cells in mice to investigate their pathogenic potential in triggering inflammation and directing the response to the optic nerves and spinal cord. As described in the Examples, AQP4 -knockout mice backcrossed to a C57BL6 background (14 crossings) were immunized with peptides corresponding to the three extracellular loops of AQP4, i.e., loop A, C and E.

It was surprisingly discovered that a robust T cell response was found only against the loop C peptide, with a remarkable T cell response, as well as development of phenotypes (e.g., paralysis from spinal cord inflammation, optic nerve inflammation) observed for the loop C sequence-containing peptide LVTPPSVVGGLGVTMVHGN (SEQ ID NO: 8); however, none of the mice produced detectable anti-AQP4 antibody. Loop C and/or Loop C sequence-containing peptide AQP4-reactive T cells were adoptively transferred to wildtype mice and within 9 days exhibited tail and hind limb weakness similar to models of experimental autoimmune encephalomyelitis (EAE) induced by T cells directed against myelin proteins. AQP4-reactive T cells polarized to a Th17 phenotype were much more likely to cause inflammation in the optic nerves in addition to the spinal cord. Histology showed demyelination, T cell infiltration and microglial activation throughout the spinal cord and optic nerve with some involvement of the brain as well. Despite widespread expression of AQP4 in other solid organs, inflammation outside of the CNS was not observed in this model. The implication of the study point to a central immunopathogenic role of AQP4-reactive T cells of NMO, specifically against the C loop peptide, notably against the Loop C sequence-containing peptide LVTPPSVVGGLGVTMVHGN (SEQ ID NO: 8), in both triggering and localizing inflammation predominantly to the optic nerves and spinal cord and suggests C loop peptides—optionally specific C loop peptide(s) such as SEQ ID NO: 8 and fragments, variants and derivatives thereof—and AQP4-reactive T cells as new treatment targets, as well as for diagnostic tests for NMO.

Accordingly, in one aspect, the present invention provides a pharmaceutical composition for treating neuromyelitis optica (NMO) comprising a therapeutically effective amount of loop C peptide of aquaporin-4 (AQP4) water channel, or a therapeutically effective fragment or variant thereof.

In yet another aspect, the invention relates to a method for treating an individual having neuromyelitis optica (NMO), comprising administering a therapeutically effective dose of loop C peptide of aquaporin-4 (AQP4) water channel, or a therapeutically effective fragment or variant thereof.

In still other aspects, the present invention provides a method for inducing a tolerance response in an individual having neuromyelitis optica (NMO), comprising administering an immunogenically effective amount of loop C peptide of aquaporin-4 (AQP4) water channel, or an immunogenic effective fragment or variant thereof.

In still other aspects, the invention relates to a pharmaceutical kit for treating an individual having neuromyelitis optica (NMO), comprising a therapeutically effective dose of loop C peptide of aquaporin-4 (AQP4) water channel, or a therapeutically effective fragment or variant thereof, and instructions for treating said individual.

In still other aspects, the invention relates to a diagnostic test for T cells that react against a loop C sequence, i.e., T cells that react against a loop C sequence-containing peptide, e.g., LVTPPSVVGGLGVTMVHGN (SEQ ID NO: 8), as a marker of NMO disease.

In certain embodiments, the loop C peptide of aquaporin-4 (AQP4) water channel is SEQ ID NO: 1 or a polypeptide having at least 90% sequence identity therewith.

In still other embodiments, the loop C peptide of aquaporin-4 (AQP4) water channel is SEQ ID NO: 1 or a polypeptide having at least 50%, or at least 55%, or at least 60%, or at least 70%, or at least 75%, or at least 80%, or at least 85%, or at least 90%, or at least 95%, or at least 99% sequence identity therewith.

In other embodiments, the loop C peptide of aquaporin-4 (AQP4) water channel is a variant of SEQ ID NO: 1.

In one aspect, the invention provides a pharmaceutical composition for treating neuromyelitis optica (NMO) comprising a therapeutically effective amount of a peptide of 50 or fewer amino acids in length that, when aligned with SEQ ID NO: 8, possesses 17 or more amino acid residues of SEQ ID NO: 8.

In yet another aspect, the invention relates to a method for treating an individual having neuromyelitis optica (NMO), comprising administering a therapeutically effective dose of a peptide of 50 or fewer amino acids in length that, when aligned with SEQ ID NO: 8, possesses 17 or more amino acid residues of SEQ ID NO: 8.

In still other aspects, the present invention provides a method for inducing a tolerance response in an individual having neuromyelitis optica (NMO), comprising administering an immunogenically effective amount of a peptide of 50 or fewer amino acids in length that, when aligned with SEQ ID NO: 8, possesses 17 or more amino acid residues of SEQ ID NO: 8.

In still other aspects, the invention relates to a pharmaceutical kit for treating an individual having neuromyelitis optica (NMO), comprising a therapeutically effective dose of a peptide of 50 or fewer amino acids in length that, when aligned with SEQ ID NO: 8, possesses 17 or more amino acid residues of SEQ ID NO: 8, and instructions for treating said individual.

In certain embodiments, the neuromyelitis optica (NMO) is monophasic neuromyelitis optica (NMO). In other embodiments, the neuromyelitis optica (NMO) is relapsing neuromyelitis optica (NMO).

In still other embodiments, the loop C sequence-containing peptide comprises 17 or more consecutive amino acid residues of SEQ ID NO: 8. Optionally, the peptide comprises SEQ ID NO: 8, or SEQ ID NO: 8 with one or two variant residues, in certain embodiments, SEQ ID NO: 8 with one variant residue. Optionally, the peptide is SEQ ID NO: 8.

In yet other embodiments, the therapeutically effective amount is sufficient to induce a tolerance response.

In other embodiments, the compositions of the invention further comprise an immunosuppression therapy. In some cases, the immunosuppression therapy can be selected from the group consisting of a glucocorticoid agent, a cytostatic agent, an antibody, an immunophilin-acting agent, an interferon, an opioid, a TNF binding protein, and a mycophenolate. In still other cases, the immunosuppression therapy can be selected from an alkylating agent, nitrogen mustard (cyclophosphamide), nitrosoureas, platinum compound, methotrexate, azathioprine, mercaptopurine, fluorouracil, protein synthesis inhibitor, dactinomycin, antracyclines, mitomycin C, bleomycin, mithramycin, IL-2 receptor-directed antibody, CD3-directed antibody, muromonab-CD3, ciclosporin (Sandimmune®), tacrolimus (Prograf®), sirolimus (Rapamune®), IFN-beta, infliximab (Remicade®), etanercept (Enbrel®), adalimumab (Humira®), fingolimod, leflunomide, rituximab (Rituxan®, MabThera®, or Zytux®), eculizumab (Soliris®), interferon beta-1a (Avonex®), natalizumab (Tysabri®), and malononitriloamides (MNA).

In another embodiment, the pharmaceutical kit of the invention further comprises an immunosuppression therapy. In some cases, the immunosuppression therapy can be selected from the group consisting of a glucocorticoid agent, a cytostatic agent, an antibody, an immunophilin-acting agent, an interferon, an opioid, a TNF binding protein, and a mycophenolate. In still other cases, the immunosuppression therapy can be selected from an alkylating agent, nitrogen mustard (cyclophosphamide), nitrosoureas, platinum compound, methotrexate, azathioprine, mercaptopurine, fluorouracil, protein synthesis inhibitor, dactinomycin, antracyclines, mitomycin C, bleomycin, mithramycin, IL-2 receptor-directed antibody, CD3-directed antibody, muromonab-CD3, ciclosporin (Sandimmune®), tacrolimus (Prograf®), sirolimus (Rapamune®), IFN-beta, infliximab (Remicade®), etanercept (Enbrel®), adalimumab (Humira®), fingolimod, leflunomide, rituximab (Rituxan®, MabThera®, or Zytux®), eculizumab (Soliris®), interferon beta-1a (Avonex®), natalizumab (Tysabri®), and malononitriloamides (MNA).

In certain embodiments, the loop C peptide of AQP4 and/or the peptide of 50 or fewer amino acids in length that, when aligned with SEQ ID NO: 8, possesses 17 or more amino acid residues of SEQ ID NO: 8 induces in a mouse administered the peptide a neurological symptom, optionally where the neurological symptom is paralysis from spinal cord inflammation or visual impairment from optic nerve inflammation. Optionally, the pharmaceutical composition, method, immunization composition or pharmaceutical kit of the invention includes a peptide that induces greater neurological symptoms in a mouse than SEQ ID NO: 9 induces.

Another aspect of the invention provides a method for detecting NMO in a subject, the method involving obtaining a T cell- and/or antibody-containing sample from a subject; contacting the sample with a peptide consisting of SEQ ID NO: 8 or a fragment or variant thereof, in an amount sufficient to allow for formation of a SEQ ID NO: 8-specific antibody-SEQ ID NO: 8 peptide complex or to allow for T cell activation in a SEQ ID NO: 8-specific manner; and detecting T cell activation in a specific manner or formation of a SEQ ID NO: 8-specific antibody-SEQ ID NO: 8 peptide complex, wherein T cell activation or formation of the SEQ ID NO: 8-specific antibody-SEQ ID NO: 8 peptide complex indicates that the subject has NMO, thereby detecting NMO in the subject.

In one embodiment, the T cell- and/or antibody-containing sample is a blood sample. In another embodiment, the sample is a plasma sample or other sample.

In another embodiment of the invention, a method of the invention further comprises administering an NMO therapy to the subject, optionally an immunosuppressive therapy and/or an AQP4 vaccine or immune tolerance therapy, optionally involving administration of a therapeutically effective amount of a peptide of 50 or fewer amino acids in length that, when aligned with SEQ ID NO: 8, possesses 17 or more amino acid residues of SEQ ID NO: 8.

Another aspect of the invention provides a kit for detecting NMO in a subject, the kit including a peptide of 50 or fewer amino acids in length that, when aligned with SEQ ID NO: 8, possesses 17 or more amino acid residues of SEQ ID NO: 8 and that, when contacted with a sample of a subject induces T cell activation, where T cell activation in a sequence-specific manner thereby indicates NMO in the subject, and instructions for its use.

In one embodiment, the peptide is SEQ ID NO: 8.

A further aspect of the invention provides an NMO model mouse induced by administration to the mouse of a peptide of 50 or fewer amino acids in length that, when aligned with SEQ ID NO: 8, possesses 17 or more amino acid residues of SEQ ID NO: 8 in an amount sufficient to create neurological symptoms in the mice.

Another aspect of the invention provides a method for identifying a candidate NMO therapeutic compound, involving administering a test compound to the NMO model mouse described herein, and identifying improvement of a neurological symptom of NMO in the NMO model mouse in the presence of the test compound, optionally as compared to an appropriate control, thereby identifying the test compound as a candidate NMO therapeutic. Optionally, the test compound is a small molecule, or is an antibody (including humanized forms and/or fragments thereof) or other biologic.

Where applicable or not specifically disclaimed, any one of the embodiments described herein are contemplated to be able to combine with any other one or more embodiments, even though the embodiments are described under different aspects of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1: Protein sequence of human aquaporin-4 peptide, isoform A (NBCI Reference Sequence: NP_001641.1) (SEQ ID NO: 1). The Figure indicates the amino acid sequences for Loop A (GTEKPLPV) (SEQ ID NO: 5), Loop C (TPPSVVGGLGVTMVHGNLTAG) (SEQ ID NO: 6), and Loop E (GNWENH) (SEQ ID NO: 7)

FIG. 2: Nucleotide sequence (cDNA) encoding human aquaporin-4 peptide, isoform A (NCBI Reference Sequence: NM_001650.4) (SEQ ID NO: 2).

FIG. 3: Protein sequence of human aquaporin-4 peptide, isoform B (NBCI Reference Sequence: NP_004019.1) (SEQ ID NO: 3).

FIG. 4: Nucleotide sequence (cDNA) encoding human aquaporin-4 peptide, isoform B (NCBI Reference Sequence: NM_004028.3 (SEQ ID NO: 4).

FIG. 5: Amino acid sequence alignment of AQP4 from Rat, Mouse, and Human and which shows extracellular loops A, C, and E. (See Pisani et al., J. boil. Chem., Mar. 18, 2011; 286(11): 9216-9224, which is incorporated herein by reference).

FIG. 6: Provides the amino acid sequences for AQP4 Loop A (GTEKPLPV) (SEQ ID NO: 5), Loop C (TPPSVVGGLGVTMVHGNLTAG) (SEQ ID NO: 6), and Loop E (GNWENH) (SEQ ID NO: 7), as well as for Loop C sequence-containing peptide 135-153 (LVTPPSVVGGLGVTMVHGN) (SEQ ID NO: 8).

FIGS. 7A, 7B: FIG. 7A shows a T cell proliferation assay reflecting a robust reaction to loop C sequence-containing peptide SEQ ID NO: 8 of AQP4. T cells from two control mice (wt-MOG) immunized with myelin-oligodendrocyte glycoprotein (MOG) peptide 35-55 showed proliferative activity as measured by incorporation of tritiated-thymidine only in the presence of the MOG antigen (red bars). T cells from a wildtype mouse immunized with AQP4 peptides (wt-AQP4) did not react against any AQP4 antigen. T cells from AQP4 null mice (KO naïve) did not inherently react to AQP4 peptides unless the mice were immunized with the peptides (K01-AQP4 and K02-AQP4). Among those tested, a peptide comprising sequence of the extracellular loop C, SEQ ID NO: 8, generated a robust reaction only in T cells from AQP4 null mice immunized against the loop C sequence-containing peptide (brown bars) or a mix of AQP4 peptides which included the loop C sequence-containing peptide (yellow bars). Results shown are means+SEM of triplicate reactions. FIG. 7B shows results of an ELISPOT assay used to determine the number of IL-17 and interferon-gamma (IFN-γ) cytokine-producing cells in Th17 polarized (Th17-pol) and unpolarized (unpol) cell cultures exposed to AQP4 loop C sequence-containing peptide (AQP4135-53) versus no stimulation (NS). Unpolarized AQP4-reactive T cells expressed significant levels of both IL-17 and IFN-γ compared to unstimulated controls. After polarization to a Th17 phenotype, the number of IL-17 producing cells almost doubled while the number of IFN-γ producing cells was nearly undetectable. However, the frequency of IL-17-producing cells also increased in the unstimulated culture (while IFN-γ-producing cells remained low).

FIGS. 8A and 8B: FIG. 8A provides behavioral assessment of adoptive intravenous transfer of T cells from AQP4 null mice immunized against loop C peptide of AQP4. The Behavior Score is a 5 point EAE-scale scoring the extent of neurologic disability (0 is no disability, 5 is death). Wildtype mice (triangles) adoptively transferred with cultured AQP4-restimulated, Th17 polarized AQP4-reactive T cells developed weakness in the tail and hind limbs (EAE score of 1.0-2.0, n=4). Transfer of AQP4-reactive T cells that were not re-stimulated with AQP4 peptide (squares, n=5) or stimulated with non-specific proteins (circles, n=6) showed no behavioral phenotype. FIG. 8B shows daily weights that demonstrated typical weight loss in mice that received the Th17 polarized AQP4-reactive and AQP4-restimulated T cells (triangles), but not in mice that received unstimulated (circles) or non-AQP4-specifically re-stimulated T cells (squares).

FIGS. 9A to 9L: Show histology of tissues from wildtype mice that received unpolarized AQP4-reactive T cells (FIGS. 9A, 9D, 9G, 9J) versus wildtype mice that received Th17-polarized AQP4-reactive T cells (FIGS. 9B, 9C, 9E, 9F, 9H, 9I, 9K, 9L). FIG. 9A shows that spinal cord parenchyma stained for CD3+ T cells showed rare, scattered cell (arrow), as compared to FIG. 9B spinal cord sections from wildtype recipients of Th17-polarized AQP4-reactive T cells, which showed intense perivascular CD3+ T cell infiltrates. FIG. 9C shows areas of demyelination (arrow shaded region) within white matter tracts (surrounding) were visible within inflammatory lesions. FIG. 9D shows that longitudinal sections of optic nerves stained for CD3+ T cells showed rare, scattered cell (arrow), compared to FIG. 9E optic nerve sections from wildtype recipients of Th17-polarized AQP4-reactive T cells, which showed intense perivascular CD3+ T cell infiltrates (arrow) FIG. 9F shows areas of demyelination (arrow pointing to shading) within white matter tracts (surrounding) were visible within inflammatory lesions. FIG. 9G shows that brain parenchyma stained for CD3+ T cells showed rare, scattered cell (arrow), compared to FIG. 9H, which shows a brain section from wildtype recipients of Th17-polarized AQP4-reactive T cells which demonstrated intense CD3+ T cell infiltrates, such as this lesion around the 3rd ventricle (arrow). FIG. 9I shows that AQP4-reactive T cells did not appear to change AQP4 staining either in lesions or in normal appearing spinal cord, optic nerve or brain, despite widespread inflammation and demyelination (representative sections from spinal cord are shown). FIG. 9J shows that despite expression of AQP4 in solid organs, rare AQP4-reactive CD3+ T cells appeared scattered throughout these organs both in the unpolarized and Th17-polarized wildtype recipients. Lung from unpolarized shown here with arrow pointing to CD3+ cells. FIG. 9K shows a lung section from Th17-polarized recipient that showed normal lung with occasional CD3+ cells (arrow). FIG. 9L shows that muscle from Th17 polarized mice showed no evidence of inflammation (arrow pointing to rare CD3+ T cells).

FIG. 10: Shows that blinded quantification of CD3 cells in the spinal cord (n=8), optic nerve (n=6) and brain (n=8) of wildtype recipients of Th17-polarized AQP4-reactive T cells demonstrated >5-fold more immunoreactivity (**p<0.01) in these tissues, as compared to unpolarized mice.

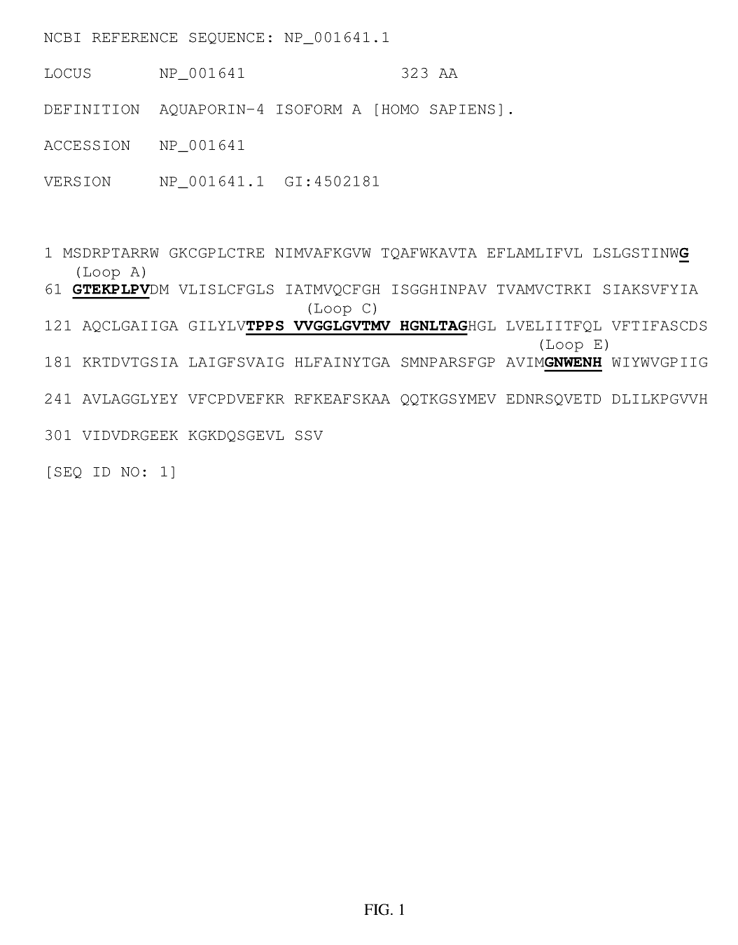

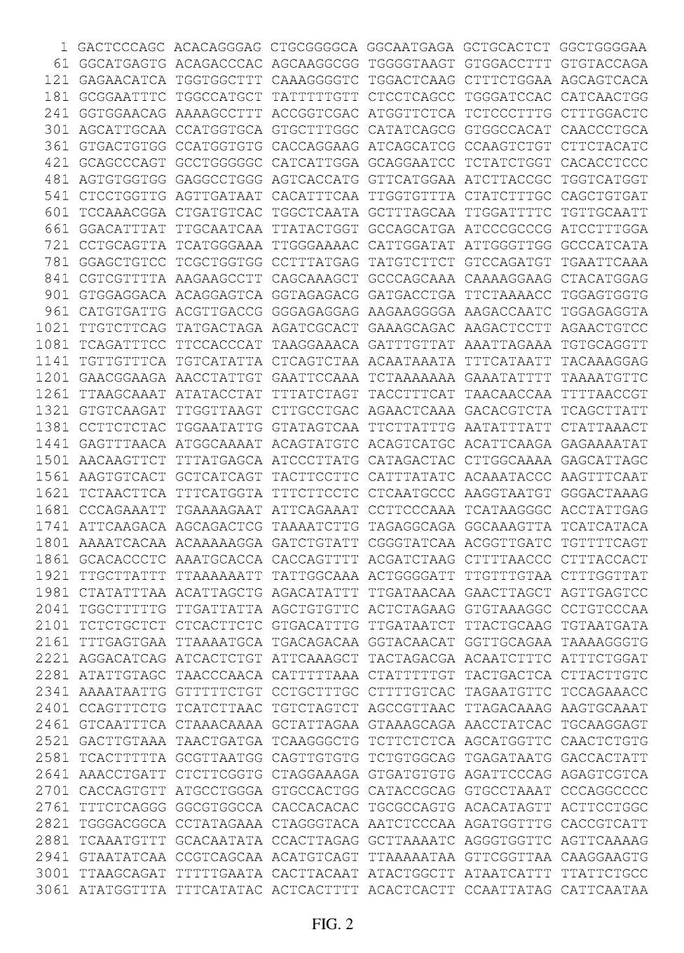

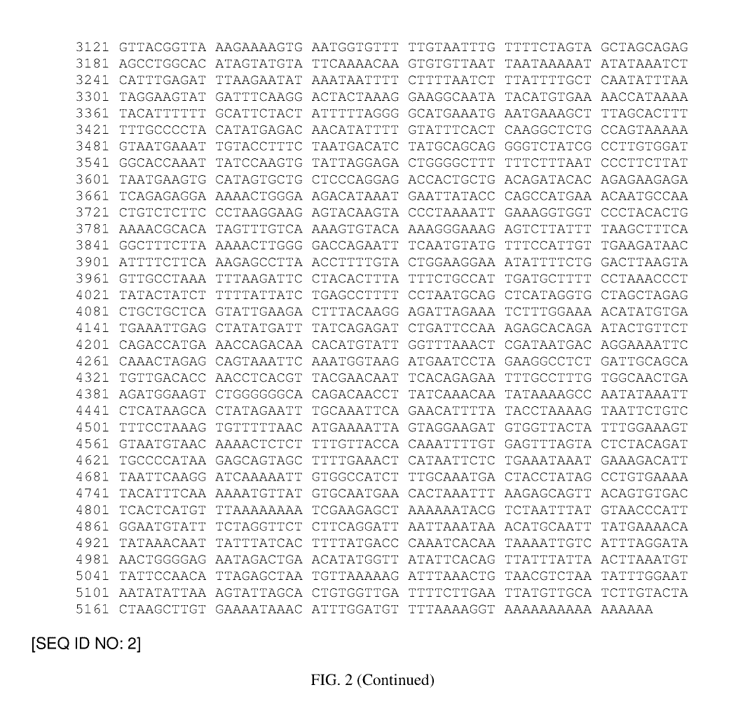

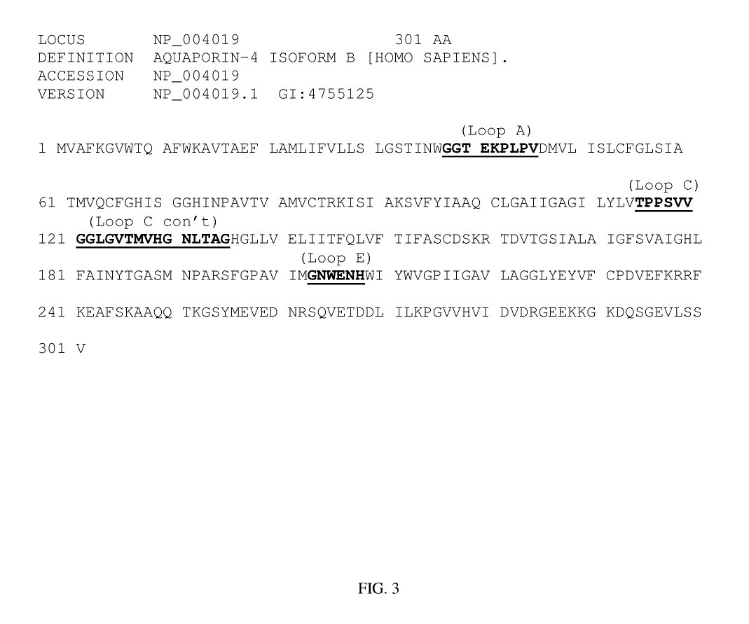

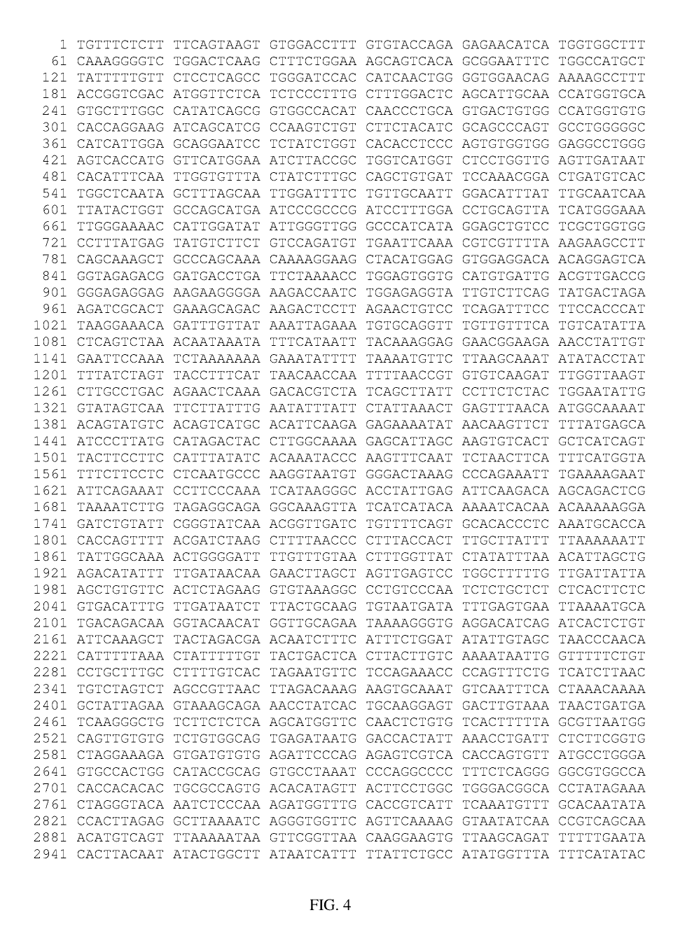

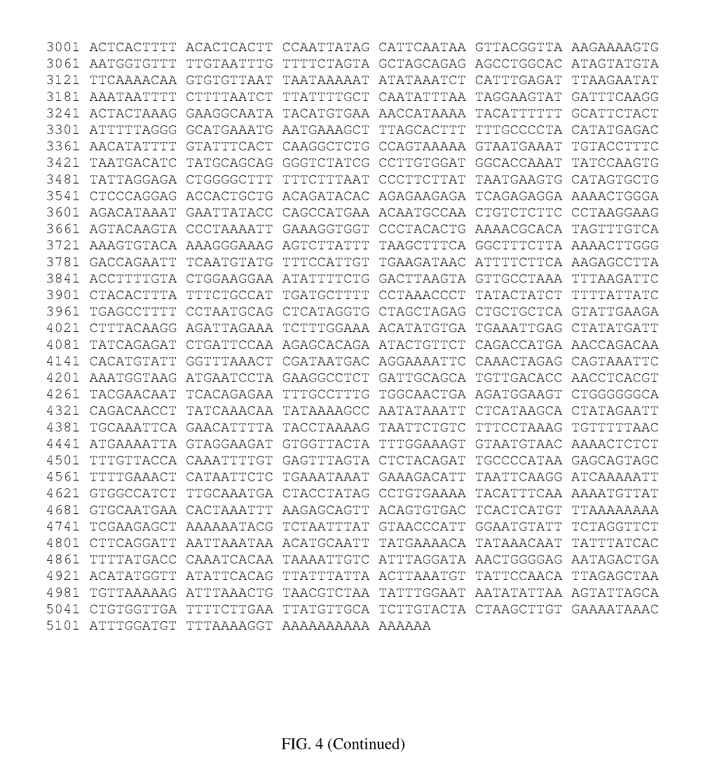

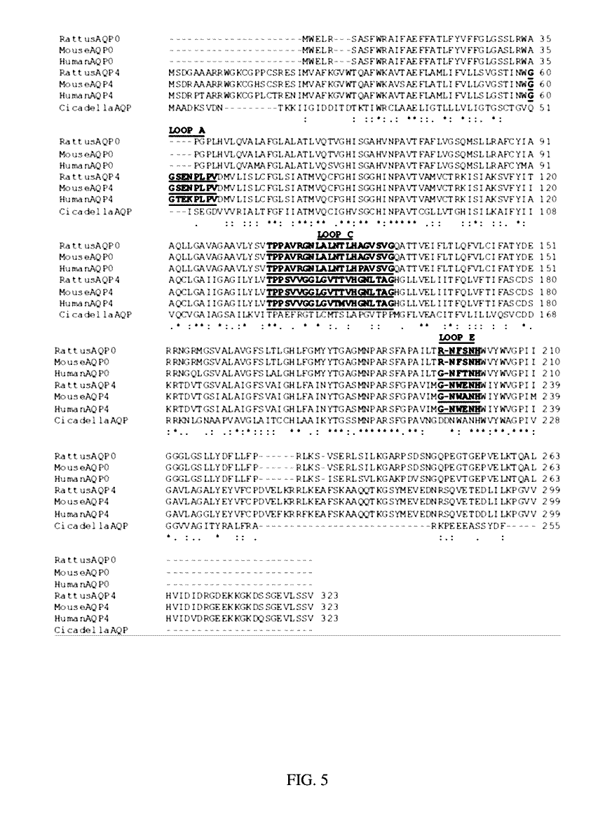

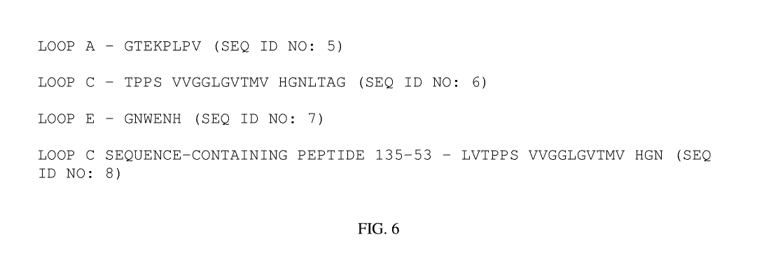

### DETAILED DESCRIPTION OF THE INVENTION

The present invention relates, in part, to the identification of a unique aquaporin-4 peptide (loop C), and particularly a loop C sequence-containing peptide SEQ ID NO: 8, that is able to trigger T cell production in AQP4-knockout mice. This surprising and previously unknown observation forms the basis of antigen-based peptide drug design for the treatment and/or amelioration of neuromyelitis optica (NMO). Accordingly, the present invention relates to pharmaceutical compositions for immunizing and/or inducing tolerance in individuals having NMO. In addition, the invention relates to methods of treating and/or immunizing NMO-patients by administering or immunizing the subjects with therapeutically effective amounts of the loop C and/or loop C sequence-containing peptide, or therapeutically effective variants and/or fragments thereof. Treatment may optionally be conducted under a setting of immunosuppression with a drug, such as, rituximab, or as a monotherapy. Administration may be by any suitable means, including oral, intravenous, subcutaneous, or intraarterial. Immunization is also contemplated by way of using nucleic acid molecules encoding the loop C and/or loop C sequence-containing peptide. Thus, the invention also pertains to nucleic acid molecules encoding the loop C and/or loop C sequence-containing peptide, and to formulations and pharmaceutical compositions for administering such molecules such that they are expressed as antigens in the individual.

As described in the Examples, in an effort to generate an accurate animal model of neuromyelitis optica (NMO), the inventors found that aquaporin-4 (AQP4) knockout mice immunized with various extracellular peptides of AQP4 responded vigorously to a single peptide that corresponds to the second extracellular loop of AQP4, loop C, with remarkable response noted to loop C sequence-containing peptide SEQ ID NO: 8. These T cells did not cause disease in AQP4 knockout mice, as these mice lacked the AQP4 target. However, when adoptively transferred to wild-type mice, these AQP4-reactive T cells caused symptomatic optic neuritis and transverse myelitis. These results highlighted the observation that loop C, and particularly loop C sequence-containing peptide SEQ ID NO: 8, was unique among AQP4 peptides in triggering a T cell reaction in AQP4 knockouts on a C57BL/6 mouse, which could mount an inflammatory attack in the spinal cord and optic nerves in wildtype C57BL/6 mice. Pathologic evaluation of spinal cord and optic nerve tissue identified a robust meningeal reaction with lymphocytes with resulting in mild neurologic symptoms. When these cells were polarized to a Th17 phenotype, the resulting phenotype was a more severe inflammatory reaction that involved the parenchyma of the optic nerves and spinal cord in a pattern similar to human NMO. The implications of this study pointed to a central immunopathogenic role of AQP4-reactive T cells of NMO in both triggering and localizing inflammation predominantly to the optic nerves and spinal cord, and thus, a new target for treating NMO was identified. Without wishing to be bound by theory, a susceptible person may be treated by exposing them to a peptide corresponding to loop C and/or a loop C sequence-containing peptide such as SEQ ID NO: 8 of AQP4, or a fragment, variant or derivative thereof, under conditions that stimulate both an auto-reactive T cell and antibody response. A Th17 response to AQP4 may cause a more fulminant disease as demonstrated in the Examples. Once AQP4-reactive T cells trigger inflammation directed to the optic nerves and spinal cord, anti-AQP4 exacerbates the pathology by fueling complement activation and granulocyte recruitment. This model identifies a new treatment target for NMO. As a disease with a highly specific antigen (AQP4) and antibody response (anti-AQP4), and now likely associated with AQP4-reactive T cells, NMO can be treated and/or ameliorated with an antigen-specific therapy in accordance with the invention. To induce a tolerance response, high dose soluble loop C and/or a loop C sequence-containing peptide may be provided to patients in the setting of immunosuppression with a drug like rituximab, commonly used to treat NMO currently. With pre-existing disease, an oral route to achieve mucosal tolerance may be the safest initial approach to avoid worsening the disease. Disease activity mediated by AQP4-reactive T cells could be mitigated by prior immunization with loop C and/or a loop C sequence-containing peptide of AQP4. Thus, the present invention relates to antigen-mediated tolerogenic approach to the treatment of NMO. Using highly soluble loop C and/or a loop C sequence-containing peptide delivered orally, intravenously or subcutaneously, either in the context of immunosuppression or as monotherapy, NMO disease in humans may be ameliorated.

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

### Definitions

Unless defined otherwise, all technical and scientific terms used herein have the meaning commonly understood by a person skilled in the art to which this invention belongs. The following references, the entire disclosures of which are incorporated herein by reference, provide one of skill with a general definition of many of the terms (unless defined otherwise herein) used in this invention: Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, the Harper Collins Dictionary of Biology (1991). Generally, the procedures of molecular biology methods described or inherent herein and the like are common methods used in the art. Such standard techniques can be found in reference manuals such as for example Sambrook et al., (2000, Molecular Cloning—A Laboratory Manual, Third Edition, Cold Spring Harbor Laboratories); and Ausubel et al., (1994, Current Protocols in Molecular Biology, John Wiley & Sons, New-York).

The following terms may have meanings ascribed to them below, unless specified otherwise. However, it should be understood that other meanings that are known or understood by those having ordinary skill in the art are also possible, and within the scope of the present invention. All publications, patent applications, patents, and other references mentioned herein are incorporated by reference in their entirety. In the case of conflict, the present specification, including definitions, will control. In addition, the materials, methods, and examples are illustrative only and not intended to be limiting.

As used herein, the singular forms “a”, “and”, and “the” include plural references unless the context clearly dictates otherwise. All technical and scientific terms used herein have the same meaning.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, the term “antigen” refers to a molecule, e.g., a peptide, polypeptide, protein, fragment, or other biological moiety, which elicits an antibody response in a subject, or is recognized and bound by an antibody, e.g., the loop C peptide of SEQ ID NO: 6 (i.e., loop C extracellular domain of AQP4).

As used herein, the term “biomarker” is understood to mean a measurable characteristic that reflects in a quantitative or qualitative manner the physiological state of an organism. The physiological state of an organism is inclusive of any disease or non-disease state, e.g., a subject having NMO or a subject who is otherwise healthy. Said another way, biomarkers are characteristics that can be objectively measured and evaluated as indicators of normal processes, pathogenic processes, or pharmacologic responses to a therapeutic intervention. Biomarkers can be clinical parameters (e.g., age, performance status), laboratory measures (e.g., molecular biomarkers, such as prostate specific antigen), imaging-based measures, or genetic or other molecular determinants, such as phosphorylation or acetylation state of a protein marker, methylation state of nucleic acid, or any other detectable molecular modification to a biological molecule. Examples of biomarkers include, for example, polypeptides, peptides, polypeptide fragments, proteins, antibodies, hormones, polynucleotides, RNA or RNA fragments, microRNA (miRNAs), lipids, polysaccharides, and other bodily metabolites.

Preferably, a biomarker of the present invention is modulated (e.g., increased or decreased level) in a biological sample from a subject or a group of subjects having a first phenotype (e.g., having a disease) as compared to a biological sample from a subject or group of subjects having a second phenotype (e.g., not having the disease, e.g., a control). A biomarker may be differentially present at any level, but is generally present at a level that is increased relative to normal or control levels by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased relative to normal or control levels by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (e.g., a p-value less than 0.05 and/or a q-value of less than 0.10 as determined using either Welch's T-test or Wilcoxon's rank-sum Test).

As used herein, the term “biopsy” or “biopsy tissue” refers to a sample of tissue (e.g., NMO lesion) that is removed from a subject for the purpose of determining if the sample contains diseased tissue. The biopsy tissue is then examined (e.g., by microscopy) for the presence or absence of the disease.

As used herein, the term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “control sample,” as used herein, refers to any clinically relevant comparative sample, including, for example, a sample from a healthy subject not afflicted with NMO, or a sample from a subject from an earlier time point, e.g., prior to treatment, an earlier drug assessment time point, at an earlier stage of treatment. A control sample can be a purified sample, protein, and/or nucleic acid provided with a kit. Such control samples can be diluted, for example, in a dilution series to allow for quantitative measurement of levels of analytes, e.g., markers, in test samples. A control sample may include a sample derived from one or more subjects. A control sample may also be a sample made at an earlier time point from the subject to be assessed. For example, the control sample could be a sample taken from the subject to be assessed before the onset of an oncological disorder, e.g., prostate cancer, at an earlier stage of disease, or before the administration of treatment or of a portion of treatment. The control sample may also be a sample from an animal model, or from a tissue or cell line derived from the animal model of neurological disorder, e.g., NMO. The level of activity or expression of one or more markers (e.g., 1, 2, 3, 4, 5, 6, 7, 8, or 9 or more markers) in a control sample consists of a group of measurements that may be determined, e.g., based on any appropriate statistical measurement, such as, for example, measures of central tendency including average, median, or modal values. Different from a control is preferably statistically significantly different from a control.

As used herein, “detecting”, “detection”, “determining”, and the like are understood to refer to an assay performed for identification of gene, protein, or peptide of the invention.

As used herein, the term “DNA” or “RNA” molecule or sequence (as well as sometimes the term “oligonucleotide”) refers to a molecule comprised generally of the deoxyribonucleotides adenine (A), guanine (G), thymine (T) and/or cytosine (C). In “RNA”, T is replaced by uracil (U).

The terms “disorders”, “diseases”, and “abnormal state” are used inclusively and refer to any deviation from the normal structure or function of any part, organ, or system of the body (or any combination thereof). A specific disease is manifested by characteristic symptoms and signs, including biological, chemical, and physical changes, and is often associated with a variety of other factors including, but not limited to, demographic, environmental, employment, genetic, and medically historical factors. Certain characteristic signs, symptoms, and related factors can be quantitated through a variety of methods to yield important diagnostic information. As used herein the disorder, disease, or abnormal state is an abnormal prostate state, including benign prostate hyperplasia and cancer, particularly prostate cancer.

The term “expression” is used herein to mean the process by which a polypeptide is produced from DNA, e.g., the loop C peptide is expressed from a nucleic acid molecule encoding same. The process involves the transcription of the gene into mRNA and the translation of this mRNA into a polypeptide. Depending on the context in which used, “expression” may refer to the production of RNA, or protein, or both.

As used herein, the term “hybridization,” as in “nucleic acid hybridization,” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 2000, supra and Ausubel et al., 1994, supra, or further in Higgins and Hames (Eds.) “Nucleic acid hybridization, a practical approach” IRL Press Oxford, Washington D.C., (1985)) and are commonly known in the art. In the case of a hybridization to a nitrocellulose filter (or other such support like nylon), as for example in the well-known Southern blotting procedure, a nitrocellulose filter can be incubated overnight at a temperature representative of the desired stringency condition (60-65° C. for high stringency, 50-60° C. for moderate stringency and 40-45° C. for low stringency conditions) with a labeled probe in a solution containing high salt (6×SSC or 5×SSPE), 5×Denhardt's solution, 0.5% SDS, and 100 μg/ml denatured carrier DNA (e.g., salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The salt and SDS concentration of the washing solutions may also be adjusted to accommodate for the desired stringency. The selected temperature and salt concentration is based on the melting temperature (Tm) of the DNA hybrid. Of course, RNA-DNA hybrids can also be formed and detected. In such cases, the conditions of hybridization and washing can be adapted according to well-known methods by the person of ordinary skill Stringent conditions are optionally used (Sambrook et al., 2000, supra). Other protocols or commercially available hybridization kits (e.g., ExpressHyb® from BD Biosciences Clonetech) using different annealing and washing solutions can also be used as well known in the art. As is well known, the length of the probe and the composition of the nucleic acid to be determined constitute further parameters of the hybridization conditions. Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Hybridizing nucleic acid molecules also comprise fragments of the above described molecules. Furthermore, nucleic acid molecules which hybridize with any of the aforementioned nucleic acid molecules also include complementary fragments, derivatives and allelic variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed).

As used herein, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95% identity), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art. Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul Nucl. Acids Res. 25 (1977), 3389-3402). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff Proc. Natl. Acad. Sci., USA, 89, (1989), 10915) uses alignments (B) of 50, expectation (E) of 10, M=5, N=4, and a comparison of both strands. Moreover, the present invention also relates to nucleic acid molecules the sequence of which is degenerate in comparison with the sequence of an above-described hybridizing molecule. When used in accordance with the present invention the term “being degenerate as a result of the genetic code” means that due to the redundancy of the genetic code different nucleotide sequences code for the same amino acid. The present invention also relates to nucleic acid molecules which comprise one or more mutations or deletions, and to nucleic acid molecules which hybridize to one of the herein described nucleic acid molecules, which show (a) mutation(s) or (a) deletion(s).

The term “modulation” refers to upregulation (i.e., activation or stimulation), down-regulation (i.e., inhibition or suppression) of a response (e.g., level of expression of a marker), or the two in combination or apart. A “modulator” is a compound or molecule that modulates, and may be, e.g., an agonist, antagonist, activator, stimulator, suppressor, or inhibitor.

As used herein, “nucleic acid molecule” or “polynucleotides”, refers to a polymer of nucleotides. Non-limiting examples thereof include DNA (e.g., genomic DNA, cDNA), RNA molecules (e.g., mRNA) and chimeras thereof, e.g., encoding the loop C peptide of SEQ ID NO: 6. The nucleic acid molecule can be obtained by cloning techniques or synthesized. DNA can be double-stranded or single-stranded (coding strand or non-coding strand [antisense]). Conventional ribonucleic acid (RNA) and deoxyribonucleic acid (DNA) are included in the term “nucleic acid” and polynucleotides as are analogs thereof. A nucleic acid backbone may comprise a variety of linkages known in the art, including one or more of sugar-phosphodiester linkages, peptide-nucleic acid bonds (referred to as “peptide nucleic acids” (PNA); Hydig-Hielsen et al., PCT Intl Pub. No. WO 95/32305), phosphorothioate linkages, methylphosphonate linkages or combinations thereof. Sugar moieties of the nucleic acid may be ribose or deoxyribose, or similar compounds having known substitutions, e.g., 2′ methoxy substitutions (containing a 2′-O-methylribofuranosyl moiety; see PCT No. WO 98/02582) and/or 2′ halide substitutions. Nitrogenous bases may be conventional bases (A, G, C, T, U), known analogs thereof (e.g., inosine or others; see The Biochemistry of the Nucleic Acids 5-36, Adams et al., ed., 11th ed., 1992), or known derivatives of purine or pyrimidine bases (see, Cook, PCT Int'l Pub. No. WO 93/13121) or “abasic” residues in which the backbone includes no nitrogenous base for one or more residues (Arnold et al., U.S. Pat. No. 5,585,481). A nucleic acid may comprise only conventional sugars, bases and linkages, as found in RNA and DNA, or may include both conventional components and substitutions (e.g., conventional bases linked via a methoxy backbone, or a nucleic acid including conventional bases and one or more base analogs). An “isolated nucleic acid molecule”, as is generally understood and used herein, refers to a polymer of nucleotides, and includes, but should not limited to DNA and RNA. The “isolated” nucleic acid molecule is purified from its natural in vivo state, obtained by cloning or chemically synthesized.

As used herein, the term “obtaining” is understood herein as manufacturing, purchasing, or otherwise coming into possession of.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthesized chemically or derived by cloning according to well-known methods. While they are usually in a single-stranded form, they can be in a double-stranded form and even contain a “regulatory region”. They can contain natural rare or synthetic nucleotides. They can be designed to enhance a chosen criteria like stability for example. Chimeras of deoxyribonucleotides and ribonucleotides may also be within the scope of the present invention.

As used herein, “one or more” is understood as each value 1, 2, 3, 4, 5, 6, 7, 8, 9, 10, and any value greater than 10.

The term “or” is used inclusively herein to mean, and is used interchangeably with, the term “and/or,” unless context clearly indicates otherwise. For example, as used herein, filamin B or LY9 is understood to include filamin B alone, LY9 alone, and the combination of filamin B and LY9.

As used herein, “patient” or “subject” can mean either a human or non-human animal, preferably a mammal having a disorder, e.g., NMO. By “subject” is meant any animal, including horses, dogs, cats, pigs, goats, rabbits, hamsters, monkeys, guinea pigs, rats, mice, lizards, snakes, sheep, cattle, fish, and birds. A human subject may be referred to as a patient. It should be noted that clinical observations described herein were made with human subjects and, in at least some embodiments, the subjects are human.

As used herein, “prophylactic” or “therapeutic” treatment refers to administration to the subject of one or more agents or interventions to provide the desired clinical effect. If it is administered prior to clinical manifestation of the unwanted condition (e.g., disease or other unwanted state of the host animal) then the treatment is prophylactic, i.e., it protects the host against developing at least one sign or symptom of the unwanted condition, whereas if administered after manifestation of the unwanted condition, the treatment is therapeutic (i.e., it is intended to diminish, ameliorate, or maintain at least one sign or symptom of the existing unwanted condition or side effects therefrom).

As use herein, the phrase “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody.

The term “such as” is used herein to mean, and is used interchangeably, with the phrase “such as but not limited to.”

The term “therapeutic effect” refers to a local or systemic effect in animals, particularly mammals, and more particularly humans caused by a pharmacologically active substance. The term thus means any substance intended for use in the diagnosis, cure, mitigation, treatment, or prevention of disease, or in the enhancement of desirable physical or mental development and conditions in an animal or human. A therapeutic effect can be understood as a decrease in tumor growth, decrease in tumor growth rate, stabilization or decrease in tumor burden, stabilization or reduction in tumor size, stabilization or decrease in tumor malignancy, increase in tumor apoptosis, and/or a decrease in tumor angiogenesis.

As used herein, “therapeutically effective amount” means the amount of a compound that, when administered to a patient for treating a disease, is sufficient to effect such treatment for the disease, e.g., the amount of such a substance that produces some desired local or systemic effect at a reasonable benefit/risk ratio applicable to any treatment, e.g., is sufficient to ameliorate at least one sign or symptom of the disease, e.g., to prevent progression of the disease or condition, e.g., prevent progression of NMO lesions or even ameliorate NMO entirely. When administered for preventing a disease, the amount is sufficient to avoid or delay onset of the disease. The “therapeutically effective amount” will vary depending on the compound, its therapeutic index, solubility, the disease and its severity and the age, weight, etc., of the patient to be treated, and the like. For example, certain compounds discovered by the methods of the present invention may be administered in a sufficient amount to produce a reasonable benefit/risk ratio applicable to such treatment. Administration of a therapeutically effective amount of a compound may require the administration of more than one dose of the compound.

A “transcribed polynucleotide” or “nucleotide transcript” is a polynucleotide (e.g. an mRNA, hnRNA, a cDNA, or an analog of such RNA or cDNA) which is complementary to or having a high percentage of identity (e.g., at least 80% identity) with all or a portion of a mature mRNA made by transcription of a marker of the invention and normal post-transcriptional processing (e.g. splicing), if any, of the RNA transcript, and reverse transcription of the RNA transcript.

“Antigenic fragment” and the like are understood as at least that portion of a peptide (e.g., loop C peptide) capable of inducing an immune response in a subject, or being able to be bound by an autoantibody present in a subject having or suspected of having an autoimmune disease, particularly NMO, particularly when the antigen is derived from AQP-4. It is understood that the peptide may not be able to induce an immune response in a normal (e.g., free from autoimmune disease) subject. However, such an antigen can promote an immune response in an animal that does not recognize the peptide as a self antigen, or who has dysfunctional immune system such that the antigen is not recognized as self. Typically, antigenic fragments are at least 7 amino acids in length. Moreover, common epitopes for autoantigens have been mapped and can be used as antigenic fragments in the compositions and methods provided herein. Antigenic fragments can include deletions of the amino acid sequence from the N-terminus or the C-terminus, or both. For example, an active fragment can have an N- and/or a C-terminal deletion of about 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, or more amino acids, depending on the starting length of the fragment. Antigenic fragments can also include one or more internal deletions of the same exemplary lengths. Antigenic fragments can also include one or more point mutations, particularly conservative point mutations. At least an antigenic fragment of an enzyme can include the full length, wild-type sequence of the antigen.

As used herein, “kits” are understood to contain at least one non-standard laboratory reagent for use in the methods of the invention in appropriate packaging, optionally containing instructions for use. The kit can further include any other components required to practice the method of the invention, as dry powders, concentrated solutions, or ready to use solutions. In some embodiments, the kit comprises one or more containers that contain reagents for use in the methods of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

A “polypeptide” or “peptide” as used herein is understood as two or more independently selected natural or non-natural amino acids joined by a covalent bond (e.g., a peptide bond). A peptide can include 2, 3, 4, 5, 6, 7, 8, 9, 10, 11, 12, 13, 14, 15, 16, 17, 18, 19, 20, or more natural or non-natural amino acids joined by peptide bonds. Polypeptides as described herein include full length proteins (e.g., fully processed proteins) as well as shorter amino acids sequences (e.g., fragments of naturally occurring proteins or synthetic polypeptide fragments).

“Sensitivity and specificity” are statistical measures of the performance of a binary classification test. The sensitivity (also called recall rate in some fields) measures the proportion of actual positives which are correctly identified as such (e.g. the percentage of sick people who are identified as having the condition); and the specificity measures the proportion of negatives which are correctly identified (e.g. the percentage of well people who are identified as not having the condition). They are closely related to the concepts of type I and type II errors. A theoretical, optimal prediction can achieve 100% sensitivity (i.e. predict all people from the sick group as sick) and 100% specificity (i.e. not predict anyone from the healthy group).

The concepts are expressed mathematically as follows:

sensitivity=# true positives/# true positives+# false negatives

specificity=# true negatives/# true negatives+# false positives.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Reference will now be made in detail to exemplary embodiments of the invention. While the invention will be described in conjunction with the exemplary embodiments, it will be understood that it is not intended to limit the invention to those embodiments. To the contrary, it is intended to cover alternatives, modifications, and equivalents as may be included within the spirit and scope of the invention as defined by the appended claims.

### Peptides and Nucleic Acids Encoding Same

In various embodiments, the compositions, methods, and kits of the invention may comprise AQP4 peptides, and in particular, peptides corresponding to the extracellular loop regions of the full protein, i.e., loop C peptide (SEQ ID NO: 6) and/or a loop C sequence-containing peptide (SEQ ID NO: 8). In other embodiments, the compositions, methods, and kits of the invention can also comprise other peptides of AQP4, including for example loop A peptide (SEQ ID NO: 5) and loop E peptide (SEQ ID NO: 7).

In still other embodiments, the compositions, methods, and kits of the invention may comprise nucleic acid molecules that encode AQP4 peptides, and in particular, peptides corresponding to the extracellular loop regions of the full protein, i.e., a loop C sequence-containing peptide (SEQ ID NO: 8). In other embodiments, the compositions, methods, and kits of the invention can also comprise nucleic acid molecules encoding other peptides of AQP4, including for example loop A peptide (SEQ ID NO: 5) and loop E peptide (SEQ ID NO: 7).

The peptides and nucleic acid molecules encoding may be used in the various methods of the invention to immunize and/or treat an individual having NMO.

Thus, in one aspect, the description provides an isolated loop C and/or loop C sequence-containing peptide (or fragment or derivative thereof) corresponding to SEQ ID NO: 6 or SEQ ID NO: 8, or a polypeptide that has at least 30%, 40%, 50%, 60%, 70%, 80%, 90% or 100% sequence identity to the polypeptide disclosed in SEQ ID NO: 6 or SEQ ID NO: 8. In other aspects, the description provides an isolated loop A peptide (or fragment or derivative thereof) corresponding to SEQ ID NO: 5, or a polypeptide that has at least 30%, 40%, 50%, 60%, 70%, 80%, 90% or 100% sequence identity to the polypeptide disclosed in SEQ ID NO:5. In still other aspects, the description provides an isolated loop E peptide (or fragment or derivative thereof) corresponding to SEQ ID NO: 7, or a polypeptide that has at least 30%, 40%, 50%, 60%, 70%, 80%, 90% or 100% sequence identity to the polypeptide disclosed in SEQ ID NO:7.

In still further aspects, the description provides an isolated loop C and/or loop C sequence-containing peptide-encoding nucleic acid molecules encoding loop C and/or loop C sequence-containing peptide (or fragment or derivative thereof) corresponding to SEQ ID NO: 6 or SEQ ID NO: 8, or a polypeptide that has at least 30%, 40%, 50%, 60%, 70%, 80%, 90% or 100% sequence identity to the polypeptide disclosed in SEQ ID NO: 6 or SEQ ID NO: 8. In other aspects, the description provides an isolated loop A peptide-enconding nucleic acid molecule encoding loop A (or fragment or derivative thereof) corresponding to SEQ ID NO: 5, or a polypeptide that has at least 30%, 40%, 50%, 60%, 70%, 80%, 90% or 100% sequence identity to the polypeptide disclosed in SEQ ID NO:5. In still other aspects, the description provides an isolated loop E peptide-encoding nucleic acid molecule encoding loop E (or fragment or derivative thereof) corresponding to SEQ ID NO: 7, or a polypeptide that has at least 30%, 40%, 50%, 60%, 70%, 80%, 90% or 100% sequence identity to the polypeptide disclosed in SEQ ID NO:7.

Thus, in one aspect, the description provides an isolated loop C and/or loop C sequence-containing peptide nucleic acid molecule encoding a loop C and/or loop C sequence-containing polypeptide that includes a nucleic acid sequence that has at least 30%, 40%, 50%, 60%, 70%, 80%, 90% or 100% identity to the nucleic acids disclosed in SEQ ID NO: 2 and 4. In certain embodiments, the isolated nucleic acid molecules of the invention will hybridize under stringent conditions to a nucleic acid sequence complementary to a nucleic acid molecule that includes a protein-coding sequence of a loop C and/or loop C sequence-containing (or loop A or loop E) nucleic acid sequence. The invention also includes an isolated nucleic acid that encodes a loop C and/or loop C sequence-containing polypeptide, or a fragment, homolog, analog, fusion protein, pseudopeptide, peptidomimetic or derivative thereof. For example, the nucleic acid can encode a polypeptide at least 30%, 40%, 50%, 60%, 70%, 80%, 90% or 100% identity to a polypeptide comprising the amino acid sequences of SEQ ID NOs. 5, 6, 7 or 8. The nucleic acid can be, for example, a genomic DNA fragment or a cDNA molecule. In certain embodiments, these loop C and/or loop C sequence-containing (or loop A or loop E) nucleic acid molecules may be modified to include certain optimization features that include modified, codon-optimized nucleotide sequences encoding loop C and/or loop C sequence-containing (or loop A or loop E), sequence tags (e.g., histidine tags, thiordoxin (Thx) tags, maltose binding protein (MBP) tags), or an additional N-terminal methionine residue, each of which result in improved and/or enhanced expression and/or solubility and/or recovery of the polypeptides of the invention.

In certain aspects, the invention provides a loop C sequence-containing peptide (or nucleic acid encoding for such peptide) that is of a shorter length than the full-length aquaporin-4 (AQP4) polypeptide (optionally, the peptide is 100 amino acid residues or less in length, 90 amino acid residues or less in length, 80 amino acid residues or less in length, 70 amino acid residues or less in length, 60 amino acid residues or less in length, 50 amino acid residues or less in length, 40 amino acid residues or less in length, 30 amino acid residues or less in length, 29 amino acid residues or less in length, 28 amino acid residues or less in length, 27 amino acid residues or less in length, 26 amino acid residues or less in length, 25 amino acid residues or less in length, 24 amino acid residues or less in length, 23 amino acid residues or less in length, 22 amino acid residues or less in length, 21 amino acid residues or less in length or 20 amino acid residues or less in length) and the peptide, when aligned with SEQ ID NO: 8 for greatest identity, includes at least 17 amino acid residues of SEQ ID NO: 8. Optionally, the peptide sequence includes at least 18 amino acid residues of SEQ ID NO: 8. In certain embodiments, the peptide sequence includes the entire sequence of SEQ ID NO: 8. In some embodiments, the peptide includes at least 17 consecutive amino acid residues of SEQ ID NO: 8; optionally, the peptide sequence includes at least 18 consecutive amino acid residues of SEQ ID NO: 8. In one embodiment, the peptide includes one or two variant residues as compared to SEQ ID NO: 8. Optionally, the peptide includes a single variant as compared to SEQ ID NO: 8 (within the SEQ ID NO: 8-aligned peptide sequence).

Optionally, the peptide of the invention is a fusion protein that includes a loop C sequence-containing peptide sequence as described herein.

In another aspect, the description provides an oligonucleotide, e.g., an oligonucleotide which includes at least 6 contiguous nucleotides of a loop C and/or loop C sequence-containing nucleic acid or a complement of said oligonucleotide.

In other aspects, the description provides substantially purified loop C and/or loop C sequence-containing polypeptides (e.g., SEQ ID NO. 6 or SEQ ID NO: 8). In certain embodiments, the loop C and/or loop C sequence-containing polypeptides include an amino acid sequence that is substantially identical to the amino acid sequence of a human loop C and/or loop C sequence-containing polypeptide. In certain other embodiments, the purified loop C and/or loop C sequence-containing polypeptides comprise at least one optimization feature, that includes a secretory signal sequence, sequence tags (e.g., histidine tags, thiordoxin (Thx) tags, maltose binding protein (MBP) tags), or an additional N-terminal methionine residue.

In still other aspects, the description provides antibodies that immunoselectively-bind to loop C and/or loop C sequence-containing polypeptides, or fragments, homologs, analogs, pseudopeptides, peptidomimetics or derivatives thereof.

In a further aspect, the description provides a method of producing a polypeptide by culturing a cell that includes an endogenous or exogenously expressed loop C and/or loop C sequence-containing nucleic acid, under conditions allowing for expression of the loop C and/or loop C sequence-containing polypeptide encoded by the DNA. If desired, the loop C and/or loop C sequence-containing polypeptide can then be recovered.

In still another aspect the description provides a method of producing a polypeptide by culturing a cell that contains an endogenous loop C and/or loop C sequence-containing nucleic acid disposed upstream or downstream of an exogenous promoter. In certain embodiments, the exogenous promoter is incorporated into a host cell's genome through homologous recombination, strand break or mismatch repair mechanisms.

In another aspect, the description provides a method of detecting the presence of a loop C and/or loop C sequence-containing polypeptide in a sample. In the method, a sample is contacted with a compound that selectively binds to the polypeptide under conditions allowing for formation of a complex between the polypeptide and the compound. The complex is detected, if present, thereby identifying the loop C and/or loop C sequence-containing polypeptide within the sample.

Also described is a method of detecting the presence of a loop C and/or loop C sequence-containing nucleic acid molecule in a sample by contacting the sample with a loop C and/or loop C sequence-containing nucleic acid probe or primer, and detecting whether the nucleic acid probe or primer bound to a loop C and/or loop C sequence-containing nucleic acid molecule in the sample.

In some embodiments of the invention, the composition may further comprise homologs, analogs, derivatives, enantiomers and/or functionally equivalent compositions thereof of the compositions of the invention. Such homologs, analogs, derivatives, enantiomers and functionally equivalent compositions thereof of the compositions may also be used in any of the assays described above. It will be understood that the skilled artisan will be able to manipulate the conditions in a manner to prepare such homologs, analogs, derivatives, enantiomers and functionally equivalent compositions. Homologs, analogs, derivatives, enantiomers and/or functionally equivalent compositions which are about as effective or more effective than the parent compound are also intended for use in the methods of the invention. Synthesis of such compositions may be accomplished through typical chemical modification methods such as those routinely practiced in the art.

Certain embodiments of the present invention involve a method comprising providing any of the compositions described herein, and performing a combinatorial synthesis on the composition, preferably to obtain homologs, analogs, derivatives, enantiomers and functionally equivalent compositions thereof of the composition. An assay may be performed with the homolog, analog, derivative, enantiomer or functionally equivalent composition to determine its effectiveness. The combinatorial synthesis can involve subjecting a plurality of the compositions described herein to combinatorial synthesis, using techniques known to those of ordinary skill in the art.

In some cases, the loop C and/or loop C sequence-containing antigens of the invention may include a hapten, i.e., a substance, typically having a low molecular weight (e.g., a small organic molecule or a peptide), which, although not capable of provoking a specific immune response when isolated by itself, is able to enhance the immune response to a chemical species (i.e., a “carrier”) to which it is attached and/or is a component of, e.g., an epitope of the antigen. The immune response may include antibodies directed against the hapten. In one set of embodiments, a portion of an antigen (e.g., an epitope) is the hapten. In another set of embodiments, the hapten is a molecule that is bound to either or both the antigen and the carbohydrate. For instance, the hapten may be a linking agent between the antigen and the carbohydrate. Non-limiting examples of haptens include certain drugs, simple sugars, amino acids, small peptides, phospholipids, triglycerides, etc.

Certain of the loop C-containing peptides of the invention (e.g., SEQ ID NO: 8) possessed effects that were demonstrably improved relative to alternative loop C-containing sequences, i.e., GILYLVTPPSVVGGLGVTMV (SEQ ID NO: 9, previously set forth in US 2014/0199333). Indeed, while phenotypes (including neurological symptoms such as paralysis from spinal cord inflammation and visual impairment from optic nerve inflammation) that were similar to NMO in human patients were observed for mice administered loop C-containing peptides of the invention (e.g., SEQ ID NO: 8), no such neurological symptoms—and by implication, correspondingly limited if any therapeutic efficacy—was observed for mice administered SEQ ID NO: 9 peptide. Thus, in certain embodiments of the invention, a loop C-containing peptide of the invention (e.g., SEQ ID NO: 8) possesses greater therapeutic efficacy in a clinical setting and/or induces greater NMO neurological symptoms in mice than other loop C sequence-containing peptides (e.g., SEQ ID NO: 9).

### Pharmaceutical Compositions

The present invention provides a pharmaceutical composition for treating neuromyelitis optica (NMO) comprising a therapeutically effective amount of loop C and/or loop C sequence-containing peptide of aquaporin-4 (AQP4) water channel, or a therapeutically effective fragment or variant thereof. In yet another aspect, the invention relates to a method for treating an individual having neuromyelitis optica (NMO), comprising administering a therapeutically effective dose of loop C and/or loop C sequence-containing peptide of aquaporin-4 (AQP4) water channel, or a therapeutically effective fragment or variant thereof. In still other aspects, the present invention provides a method for inducing a tolerance response in an individual having neuromyelitis optica (NMO), comprising administering an immunogenically effective amount of loop C and/or loop C sequence-containing peptide of aquaporin-4 (AQP4) water channel, or an immunogenic effective fragment or variant thereof.

### Formulations

Pharmaceutical compositions of the invention comprising therapeutically effective amounts of loop C and/or loop C sequence-containing peptide (or fragments or variants thereof) or therapeutically effective amounts of loop C nucleic acidsd can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified immunoresponsive cells utilized in practicing the present invention in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the genetically modified immunoresponsive cells or their progenitors.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the genetically modified immunoresponsive cells as described in the present invention. This will present no problem to those skilled in chemical and pharmaceutical principles, or problems can be readily avoided by reference to standard texts or by simple experiments (not involving undue experimentation), from this disclosure and the documents cited herein. Such determinations do not require undue experimentation from the knowledge of the skilled artisan, this disclosure and the documents cited herein. And, the time for sequential administrations can be ascertained without undue experimentation.

Immune responses induced by the compositions of the invention can also be augmented by the co-administration or co-linear expression of cytokines or B7-1/2 co-stimulatory molecules in combination with the compositions of the invention. The cytokines can be administered directly with the compositions, and/or may be administered in the form of a nucleic acid vector that encodes the cytokine, such that the cytokine can be expressed in vivo. In one embodiment, the cytokine is administered in the form of a plasmid expression vector. The term “cytokine” is used as a generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment. Non-limiting examples of cytokines include, but are not limited to IL-1, IL-2, IL-4, IL-5, IL-6, IL-7, IL-10, IL-12, IL-15, IL-18 granulocyte-macrophage colony stimulating factor (GM-CSF), granulocyte colony stimulating factor (GCSF), interferon-gamma, interferon-alpha, tumor necrosis factor-alpha, tumor necrosis factor-beta, TGF-gamma, FLT-3 ligand, CD40 ligand, etc.

In certain embodiments of the invention, the agents of the invention may be administered in conjunction with an adjuvant. An “adjuvant,” as used herein, is any molecule or compound that can stimulate the humoral and/or cellular immune response or function as a depot for the antigen. Examples of adjuvants include adjuvants that create a depot effect, immune stimulating adjuvants, adjuvants that create a depot effect and stimulate the immune system, and mucosal adjuvants.

An “adjuvant that creates a depot effect” as used herein is an adjuvant that causes an antigen to be slowly released in the body, thus prolonging the exposure of immune cells to the antigen. This class of adjuvants includes but is not limited to alum (e.g., aluminum hydroxide, aluminum phosphate), or emulsion-based formulations including mineral oil, non-mineral oil, water-in-oil or oil-in-water-in oil emulsion, oil-in-water emulsions such as Seppic ISA series of Montamide adjuvants (e.g., Montamide ISA 720, AirLiquide, Paris, France), MF-59 (a squalene-in-water emulsion stabilized with Span 85 and Tween 80, Chiron Corporation), and PROVAX (an oil-in-water emulsion containing a stabilizing detergent and a micelle-forming agent, IDEC, Pharmaceuticals Corporation).

An “immune stimulating adjuvant” is an adjuvant that causes activation of a cell of the immune system. It may, for instance, cause an immune cell to produce and secrete cytokines. This class of adjuvants includes but is not limited to saponins purified from the bark of the Q. saponaria tree, such as QS21 (a glycolipid that elutes in the 21 t peak with HPLC fractionation, Aquila Biopharmaceuticals, Inc.), poly(di(carboxylatophenoxy)phosphazene) (PCPP polymer, Virus Research Institute), derivatives of lipopolysaccharides such as monophosphoryl lipid A (MPL, Ribi ImmunoChem Research, Inc.), muramyl dipeptide (MDP, Ribi) andthreonyl-muramyl dipeptide (t-MDP, Ribi), OM-174 (a glucosamine disaccharide related to lipid A, OM Pharma SA), and Leishmania elongation factor (a purified Leishmania protein, Corixa Corporation).

“Adjuvants that create a depot effect and stimulate the immune system” are those compounds which have both of the above-identified functions. This class of adjuvants includes but is not limited to ISCOMS (immunostimulating complexes which contain mixed saponins, lipids and form virus-sized particles with pores that can hold antigen, CSL), SB-AS2 (SmithKline Beecham adjuvant system #2, which is an oil-in-water emulsion containing MPL and QS21: SmithKline Beecham Biologicals), SB-AS4 (SmithKline Beecham adjuvant system #4 which contains alum and MPL), non-ionic block copolymers that form micelles such as CRL 1005 (these contain a linear chain of hydrophobic polyoxpropylene flanked by chains of polyoxyethylene, Vaxcel, Inc.), and Syntex Adjuvant Formulation (SAF, an oil-in-water emulsion containing Tween 80 and a nonionic block copolymer, Syntex Chemicals, Inc.).

A “mucosal adjuvant” as used herein is an adjuvant that is capable of inducing a mucosal immune response in a subject when administered to a mucosal surface in conjunction with an antigen. Mucosal adjuvants include but are not limited to bacterial toxins, for example, cholera toxin and cholera toxin derivatives (e.g., CT B subunit, CTD53, CTK97, CTK104, CTD53/K63, CTH54, CTN107, CTE114, CTE112K, CTS61F, CTS106, CTK63, etc.), Zonula occludens toxin, Escherichia coli heat-labile enterotoxin, labile toxin and labile toxin derivatives (e.g., LT B subunit (LTB), LT7K, LT61F, LT112K, LT118E, LT146E, LT192G, LTK63, LTR72, etc.), Pertussis toxin and Pertussis toxin derivatives (e.g., PT-9K/129G), Lipid A derivatives (e.g., monophosphoryl lipid A, MPL), muramyl dipeptide derivatives, bacterial outer membrane proteins (e.g., outer surface protein A (OspA) lipoprotein of Borrelia burgdorferi, outer membrane protein of Neisseria meningitidis, etc.), oil-in-water emulsions (e.g., MF59), aluminum salts, saponins, etc.

### NMO Targets the Spinal Cord and Optic Nerve

Neuromyelitis optica (NMO) is a relapsing autoimmune disease primarily targeting the spinal cord and optic nerve leading to paralysis and blindness (1). The discovery of the highly specific antiaquaporin4 (AQP4) IgG1 biomarker implicated an immune reaction against AQP4 evidenced by both humoral and cellular pathology within acute NMO lesions (2, 3). Several previous mouse and rat models of NMO focused on the role of the circulating anti-AQP4 antibody in disease pathogenesis concluded that the antibody by itself was insufficient to induce disease, but could exacerbate an experimental autoimmune encephalomyelitis induced by myelin-reactive T cells (4-8). When the anti-AQP4 antibody had passive access to AQP4 on astrocytes in the nervous system, there was abundant evidence that the antibody bound to AQP4 and could participate in complement-mediated damage to astrocytes under experimental conditions (9-11). Collectively, these studies indicated an important role for the anti-AQP4 antibody in enhancing astrocytic damage from NMO relapses, rather than in the triggering of NMO attacks, prompting a search for other AQP4-specific immune components that might be involved upstream in the immunopathogenesis of NMO. In each passive transfer study, the anti-AQP4 antibody was only pathogenic in the context of a T-cell-based autoimmune attack on the central nervous system.

Without wishing to be bound by theory, production of the IgG₁ biomarker against AQP4 presumably required AQP4-reactive B and T cells for immunoglobulin class switching. T cells are also among the inflammatory cells found in acute NMO lesions, and their role in the immunopathogenesis of NMO disease has been the subject of recent studies in which immunodominant AQP4 peptides were shown to trigger T cell activation in mice (12, 13). However, despite activating T cells against AQP4, these rat and mouse models did not develop a clinical neurological phenotype, either because pathogenic T cell responses were limited by a combination of central and peripheral tolerance or because certain AQP4 epitopes were not pathogenic. A unique approach was employed to raise pathogenic AQP4-reactive T cells in AQP4 null mice, which caused an NMO-like disease when adoptively transferred to wild-type mice. Polarization of AQP4-reactive T cells to the T helper17 phenotype enhanced the phenotype and led to inflammation and demyelination in the optic nerves and spinal cord, as well as the brain. There was no other evidence of solid organ inflammation despite widespread AQP4 expression in the mouse, supporting the specificity of this approach to modeling the human NMO disease.

### Administration

Compositions comprising the AQP4 peptides of the invention, or nucleic acid molecules encoding same, can be provided systemically or directly to a subject for the treatment of a neoplasia, pathogen infection, or infectious disease. In one embodiment, compositions of the invention are directly injected into an organ of interest (e.g., an organ affected by a neoplasia). Alternatively, compositions are provided indirectly to the organ of interest, for example, by administration into the circulatory system (e.g., the tumor vasculature).

The compositions can be administered in any physiologically acceptable vehicle, normally intravascularly, although they may also be introduced into bone or other convenient site where the cells may find an appropriate site for regeneration and differentiation (e.g., thymus). Usually, at least 1×10⁵ cells will be administered, eventually reaching 1×10¹⁰, or more. Dosages can be readily adjusted by those skilled in the art (e.g., a decrease in purity may require an increase in dosage). The compositions can be introduced by injection, catheter, or the like. If desired, factors can also be included, including, but not limited to, interleukins, e.g. IL-2, IL-3, IL-6, and IL-11, as well as the other interleukins, the colony stimulating factors, such as G-, M- and GM-CSF, interferons, e.g. gamma-interferon and erythropoietin.

Compositions of the invention include pharmaceutical compositions comprising polypeptides of the invention and a pharmaceutically acceptable carrier.

Compositions can be administered via localized injection, including catheter administration, systemic injection, localized injection, intravenous injection, or parenteral administration. When administering a therapeutic composition of the present invention, it will generally be formulated in a unit dosage injectable form (solution, suspension, emulsion).

Essentially, administration of a composition of the invention may be accomplished by any medically acceptable method which allows the composition to reach its target, i.e., NMO lesions of the spinal cord and optic nerves. The particular mode selected will depend of course, upon factors such as those previously described, for example, the particular composition, the severity of the state of the subject being treated, the dosage required for therapeutic efficacy, etc. As used herein, a “medically acceptable” mode of treatment is a mode able to produce effective levels of the composition within the subject without causing clinically unacceptable adverse effects.

Any medically acceptable method may be used to administer the compositions to the subject. The administration may be localized (i.e., to a particular region, physiological system, tissue, organ, or cell type) or systemic, depending on the condition to be treated. For example, the composition may be administered orally, vaginally, rectally, buccally, pulmonary, topically, nasally, transdermally, sublingually, through parenteral injection or implantation, via surgical administration, or any other method of administration where access to the target by the composition of the invention is achieved. Examples of parenteral modalities that can be used with the invention include intravenous, intradermal, subcutaneous, intracavity, intramuscular, intraperitoneal, epidural, or intrathecal. Examples of implantation modalities include any implantable or injectable drug delivery system. Oral administration may be preferred in some embodiments because of the convenience to the subject as well as the dosing schedule. Compositions suitable for oral administration may be presented as discrete units such as hard or soft capsules, pills, cachettes, tablets, troches, or lozenges, each containing a predetermined amount of the active compound. Other oral compositions suitable for use with the invention include solutions or suspensions in aqueous or non-aqueous liquids such as a syrup, an elixir, or an emulsion. In another set of embodiments, the composition may be used to fortify a food or a beverage.

In certain embodiments of the invention, the administration of the composition of the invention may be designed so as to result in sequential exposures to the composition over a certain time period, for example, hours, days, weeks, months or years. This may be accomplished, for example, by repeated administrations of a composition of the invention by one of the methods described above, or by a sustained or controlled release delivery system in which the composition is delivered over a prolonged period without repeated administrations. Administration of the composition using such a delivery system may be, for example, by oral dosage forms, bolus injections, transdermal patches or subcutaneous implants. Maintaining a substantially constant concentration of the composition may be preferred in some cases.

The composition may also be administered on a routine schedule, but alternatively, may be administered as symptoms arise. A “routine schedule” as used herein, refers to a predetermined designated period of time. The routine schedule may encompass periods of time which are identical or which differ in length, as long as the schedule is predetermined. For instance, the routine schedule may involve administration of the composition on a daily basis, every two days, every three days, every four days, every five days, every six days, a weekly basis, a bi-weekly basis, a monthly basis, a bimonthly basis or any set number of days or weeks there-between, every two months, three months, four months, five months, six months, seven months, eight months, nine months, ten months, eleven months, twelve months, etc. Alternatively, the predetermined routine schedule may involve administration of the composition on a daily basis for the first week, followed by a monthly basis for several months, and then every three months after that. Any particular combination would be covered by the routine schedule as long as it is determined ahead of time that the appropriate schedule involves administration on a certain day.

In some cases, the composition is administered to the subject in anticipation of an NMO event in order to prevent an NMO lesion event. As used herein, “substantially prior” means at least six months, at least five months, at least four months, at least three months, at least two months, at least one month, at least three weeks, at least two weeks, at least one week, at least 5 days, or at least 2 days prior to the NMO lesion event.

Similarly, the compositions may be administered immediately prior to an NMO lesion event (e.g., within 48 hours, within 24 hours, within 12 hours, within 6 hours, within 4 hours, within 3 hours, within 2 hours, within 1 hour, within 30 minutes or within 10 minutes of an NMO event), substantially simultaneously with the NMO event or following the lesion symptoms.

Other delivery systems suitable for use with the present invention include time-release, delayed release, sustained release, or controlled release delivery systems. Such systems may avoid repeated administrations of the composition in many cases, increasing convenience to the subject. Many types of release delivery systems are available and known to those of ordinary skill in the art. They include, for example, polymer-based systems such as polylactic and/or polyglycolic acids, polyanhydrides, polycaprolactones and/or combinations of these; nonpolymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include, but are not limited to, erosional systems in which the composition is contained in a form within a matrix (for example, as described in U.S. Pat. Nos. 4,452,775, 4,675,189, and 5,736,152), or diffusional systems in which an active component controls the release rate (for example, as described in U.S. Pat. Nos. 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the composition. In addition, a pump-based hardware delivery system may be used to deliver one or more embodiments of the invention.

Use of a long-term release implant may be particularly suitable in some embodiments of the invention. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

Administration of the composition can be alone, or in combination with other therapeutic agents and/or compositions, for example, used to treat allergies, infectious disease, cancers, etc. In a particular embodiment, the AQP4 peptides of the invention (or nucleic acids encoding same) are administered together with immunosuppression thereapy.

The immunosuppression therapy can be selected from the group consisting of a glucocorticoid agent, a cytostatic agent, an antibody, an immunophilin-acting agent, an interferon, an opioid, a TNF binding protein, and a mycophenolate. Immunosuppression therapy can also be an alkylating agent, nitrogen mustard (cyclophosphamide), nitrosoureas, platinum compound, methotrexate, azathioprine, mercaptopurine, fluorouracil, protein synthesis inhibitor, dactinomycin, antracyclines, mitomycin C, bleomycin, mithramycin, IL-2 receptor-directed antibody, CD3-directed antibody, muromonab-CD3, ciclosporin (Sandimmune®), tacrolimus (Prograf®), sirolimus (Rapamune®), IFN-beta, infliximab (Remicade®), etanercept (Enbrel®), adalimumab (Humira®), fingolimod, leflunomide, rituximab (Rituxan®, MabThera®, or Zytux®), eculizumab (Soliris®), interferon beta-1a (Avonex®), natalizumab (Tysabri®), and malononitriloamides (MNA).

The compositions of the invention can also be administered with other therapeutic agents and drugs that can be used in combination with one or more compositions of the invention for the treatment of other related (M.S.) or non-related indications (e.g., cancer, infections).

For instance, examples of therapeutic agents and drugs that can be used in combination with one or more compositions of the invention for the treatment of allergy include, but are not limited to, one or more of: PDE-4 inhibitors, bronchodilator (e.g., salmeterol, salbutamol, albuterol, terbutaline, D2522/formoterol, fenoterol, bitolterol, pirbuerol, methylxanthines such as theophylline, orciprenaline, etc.), beta-2 agonists (e.g., albuterol, bitolterol, pirbuterol, terbutaline, etc.), K⁺ channel openers, VLA-4 antagonists, neurokin antagonists, TXA2 synthesis inhibitors, xanthanines, arachidonic acid antagonists, 5-lipoxygenase inhibitors, thromboxin A2 receptor antagonists, thromboxane A2 antagonists, inhibitors of 5-lipox activation proteins, protease inhibitors, chromolyn sodium, or medocromil. Other examples of potentially useful allergy medicaments include, but are not limited to, loratidine, cetirizine, buclizine, ceterizine analogues, fexofenadine, terfenadine, desloratadine, norastemizole, epinastine, ebastine, ebastine, astemizole, levocabastine, azelastine, tranilast, terfenadine, mizolastine, betatastine, CS 560, HSR 609, prostaglandins, steroids (e.g., beclomethasone, fluticasone, tramcinolone, budesonide, budesonide, etc.), corticosteroids (e.g., beclomethasome dipropionate, budesonide, flunisolide, fluticaosone, propionate, triamcinoone acetonide, dexamethasone, methylprednisolone, prednisolone, prednisone etc.), immunomodulators (e.g., anti-inflammatory agents, leukotriene antagonists such as zafirlukast or zileuton, IL-4 muteins, soluble IL-4 receptors, immunosuppressants such as tolerizing peptide vaccine, anti-IL-4 antibodies, IL-4 antagonists, anti-IL-5 antibodies, soluble IL-13 receptor-Fc fusion proteins, anti-IL-9 antibodies, CCR3 antagonists, CCR5 antagonists, VLA-4 inhibitors, etc), downregulators of IgE (e.g., peptides or other molecules with the ability to bind to the IgE receptor, monoclonal antibodies against IgE, certain polypeptides capable of blocking the binding of the IgE antibody, etc.). Still other potentially useful immunomodulators include neuropeptides that have been shown to have immunomodulating properties, for example, substance P.

The term “cancer,” as used herein, may include, but is not limited to: biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma, teratomas, choriocarcinomas; stromal tumors and germ cell tumors; thyroid cancer including thyroid adenocarcinoma and medullar carcinoma; and renal cancer including adenocarcinoma and Wilms' tumor. Commonly encountered cancers include breast, prostate, lung, ovarian, colorectal, and brain cancer. In general, an effective amount of the compositions of the invention for treating cancer will be that amount necessary to inhibit mammalian cancer cell proliferation in situ. Those of ordinary skill in the art are well-schooled in the art of evaluating effective amounts of anti-cancer agents.

The term “cancer treatment” as used herein, may include, but is not limited to: chemotherapy, radiotherapy, adjuvant therapy, or any combination of these methods. Aspects of cancer treatment that may vary include, but are not limited to, dosages, timing of administration or duration or therapy; and such aspects may or may not be combined with other treatments, which may also vary in dosage, timing, and/or duration. Another cancer treatment is surgery, which may be utilized either alone or in combination with any of the previously-described treatment methods. One of ordinary skill in the medical arts can determine an appropriate cancer treatment for a subject.

Non-limiting examples of anti-cancer agents and drugs that can be used in combination with one or more compositions of the invention for the treatment of cancer include, but are not limited to, one or more of: 20-epi-1,25 dihydroxyvitamin D3, 4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caracemide, carbetimer, carboplatin, carboxamide-amino-triazole, carboxyamidotriazole, carest M3, carmustine, carn 700, cartilage derived inhibitor, carubicin hydrochloride, carzelesin, casein kinase inhibitors, castanospermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, edelfosine, edrecolomab, eflornithine, eflornithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, flurocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, O6-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RH retinamide, RNAi, rogletimide, rohitukine, romurtide, roquinimex, rubiginone B1, ruboxyl, safingol, safingol hydrochloride, saintopin, sarcnu, sarcophytol A, sargramostim, SDI 1 mimetics, semustine, senescence derived inhibitor 1, sense oligonucleotides, signal transduction inhibitors, signal transduction modulators, simtrazene, single chain antigen binding protein, sizofiran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, and zorubicin hydrochloride, as well as salts, homologs, analogs, derivatives, enantiomers and/or functionally equivalent compositions thereof.

Other examples of agents useful in the treatment of cancer include, but are not limited to, one or more of Ributaxin, Herceptin, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART 1D10 Ab, SMART ABL 364 Ab and ImmuRAIT-CEA.

An “infectious disease” as used herein, refers to a disorder arising from the invasion of a host, superficially, locally, or systemically, by an infectious microorganism. Infectious microorganisms include, but are not limited to, bacteria, viruses, fungi, molds, etc. Examples of therapeutic agents and drugs that can be used in combination with one or more compositions of the invention for the treatment of infectious disease include anti-microbial agents, antibacterial agents, antivrial agents, nucleotide analogs, antifungal agents antibiotics, etc. Such agents and/or drugs include naturally-occurring or synthetic compounds that are capable of killing or inhibiting infectious microorganisms. The type of anti-microbial agent useful according to the invention will depend upon the type of microorganism with which the subject is infected or at risk of becoming infected.

Antibiotics potentially useful in the invention include broad spectrum antibiotics and narrow spectrum antibiotics. Antibiotics that are effective against a single organism or disease and not against other types of bacteria, are generally referred to as limited spectrum antibiotics. In general, antibacterial agents are cell wall synthesis inhibitors, such as beta-lactam antibiotics (e.g., carbapenems and cephalolsporins, including cephalothin, cephapirin, cephalexin, cefamandole, cefaclor, cefazolin, cefuroxine, cefoxitin, cefotaxime, cefsulodin, cefetamet, cefixime, ceftriaxone, cefoperazone, ceftazidine, moxalactam, etc.), natural penicillins, semi-synthetic penicillins (e.g., ampicillin, carbenicillin, oxacillin, azlocillin, mezlocillin, piperacillin, methicillin, dicloxacillin, nafcillin, etc.), ampicillin, clavulanic acid, cephalolsporins, bacitracin, etc.; cell membrane inhibitors (e.g., polymyxin, amphotericin B, nystatin, imidazoles including clotrimazole, miconazole, ketoconazole, itraconazole, fluconazole, etc.); protein synthesis inhibitors (e.g., tetracyclines, chloramphenicol, macrolides such as erythromycin, aminoglycosides such as streptomycin, rifampins, ethambutol, streptomycin, kanamycin, tobramycin, amikacin, gentamicin, tetracyclines (e.g., tetracycline, minocycline, doxycycline, and chlortetracycline, etc.), erythromycin, roxithromycin, clarithromycin, oleandomycin, azithromycin, chloramphenicol, etc.); nucleic acid synthesis or functional inhibitors (e.g., quinolones, co-trimoxazole, rifamycins, etc.); competitive inhibitors (e.g., sulfonamides such as gantrisin, trimethoprim, etc.).

Antiviral agents are compounds which prevent infection of cells by viruses or replication of the virus within the cell. There are several stages within the process of viral infection which can be blocked or inhibited by antiviral agents. These stages include attachment of the virus to the host cell (e.g., immunoglobulin, binding peptides, etc.), uncoating of the virus (e.g. amantadine), synthesis or translation of viral mRNA (e.g. interferon), replication of viral RNA or DNA (e.g. nucleoside analogs), maturation of new virus proteins (e.g. protease inhibitors), budding and release of the virus, etc.

Nucleotide analogs are synthetic compounds which are similar to nucleotides, but which may have an incomplete or abnormal deoxyribose or ribose group. Nucleotide analogs include, but are not limited to, acyclovir, gancyclovir, idoxuridine, ribavirin, dideoxyinosine, dideoxycytidine, and zidovudine (azidothymidine).

Antifungal agents are useful for the treatment and prevention of infective fungi. Some anti-fungal agents function as cell wall inhibitors by inhibiting glucose synthase. These include, but are not limited to, basiungin/ECB. Other anti-fungal agents function by destabilizing membrane integrity. These include, but are not limited to, immidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, terbinafine, etc. Other anti-fungal agents function by breaking down chitin (e.g. chitinase) or immunosuppression (501 cream).

In certain embodiments of the invention, a composition can be combined with a suitable pharmaceutically acceptable carrier, for example, as incorporated into a liposome, incorporated into a polymer release system, or suspended in a liquid, e.g., in a dissolved form or a colloidal form. In general, pharmaceutically acceptable carriers suitable for use in the invention are well-known to those of ordinary skill in the art. As used herein, a “pharmaceutically acceptable carrier” refers to a non-toxic material that does not significantly interfere with the effectiveness of the biological activity of the active compound(s) to be administered, but is used as a formulation ingredient, for example, to stabilize or protect the active compound(s) within the composition before use. A pharmaceutically acceptable carrier may be sterile in some cases. The term “carrier” denotes an organic or inorganic ingredient, which may be natural or synthetic, with which one or more active compounds of the invention are combined to facilitate the application of the composition. The carrier may be co-mingled or otherwise mixed with one or more active compounds of the present invention, and with each other, in a manner such that there is no interaction which would substantially impair the desired pharmaceutical efficacy. The carrier may be either soluble or insoluble, depending on the application. Examples of well-known carriers include glass, polystyrene, polypropylene, polyethylene, dextran, nylon, amylase, natural and modified cellulose, polyacrylamide, agarose and magnetite. The nature of the carrier can be either soluble or insoluble. Those skilled in the art will know of other suitable carriers, or will be able to ascertain such, using only routine experimentation.

In some embodiments, the compositions of the invention include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers that may be used with the active compound. For example, if the formulation is a liquid, the carrier may be a solvent, partial solvent, or non-solvent, and may be aqueous or organically based. Examples of suitable formulation ingredients include diluents such as calcium carbonate, sodium carbonate, lactose, kaolin, calcium phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal. Suitable carrier concentrations can be determined by those of ordinary skill in the art, using no more than routine experimentation. The compositions of the invention may be formulated into preparations in solid, semi-solid, liquid or gaseous forms such as tablets, capsules, elixirs, powders, granules, ointments, solutions, depositories, inhalants or injectables. Those of ordinary skill in the art will know of other suitable formulation ingredients, or will be able to ascertain such, using only routine experimentation.

Preparations include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like. Those of skill in the art can readily determine the various parameters for preparing and formulating the compositions of the invention without resort to undue experimentation.

In some embodiments, the present invention includes the step of bringing a composition of the invention into association or contact with a suitable carrier, which may constitute one or more accessory ingredients. The final compositions may be prepared by any suitable technique, for example, by uniformly and intimately bringing the composition into association with a liquid carrier, a finely divided solid carrier or both, optionally with one or more formulation ingredients as previously described, and then, if necessary, shaping the product.

In some embodiments, the compositions of the present invention may be present as a pharmaceutically acceptable salt. The term “pharmaceutically acceptable salts” includes salts of the composition, prepared in combination with, for example, acids or bases, depending on the particular compounds found within the composition and the treatment modality desired. Pharmaceutically acceptable salts can be prepared as alkaline metal salts, such as lithium, sodium, or potassium salts; or as alkaline earth salts, such as beryllium, magnesium or calcium salts. Examples of suitable bases that may be used to form salts include ammonium, or mineral bases such as sodium hydroxide, lithium hydroxide, potassium hydroxide, calcium hydroxide, magnesium hydroxide, and the like. Examples of suitable acids that may be used to form salts include inorganic or mineral acids such as hydrochloric, hydrobromic, hydroiodic, hydrofluoric, nitric, carbonic, monohydrogencarbonic, phosphoric, monohydrogenphosphoric, dihydrogenphosphoric, sulfuric, monohydrogensulfuric, phosphorous acids and the like. Other suitable acids include organic acids, for example, acetic, propionic, isobutyric, maleic, malonic, benzoic, succinic, suberic, fumaric, mandelic, phthalic, benzenesulfonic, p-tolylsulfonic, citric, tartaric, methanesulfonic, glucuronic, galacturonic, salicylic, formic, naphthalene-2-sulfonic, and the like. Still other suitable acids include amino acids such as arginate, aspartate, glutamate, and the like.

### Doses

Any of the compositions of the present invention may be administered to the subject in a therapeutically effective dose. A “therapeutically effective” or an “effective” amount or dose, as used herein means that amount necessary to induce immunity or tolerance within the subject, and/or to enable the subject to more effectively mitigate the symptoms of the NMO or ameliorate NMO altogether. When administered to a subject, effective amounts will depend on the particular condition being treated and the desired outcome. A therapeutically effective dose may be determined by those of ordinary skill in the art, for instance, employing factors such as those further described below and using no more than routine experimentation.

In some embodiments, a therapeutically effective amount can be initially determined from cell culture assays. For instance the effective amount of a composition of the invention useful for inducing dendritic cell response can be assessed using the in vitro assays with respect to a stimulation index. The stimulation index can be used to determine an effective amount of a particular composition of the invention for a particular subject, and the dosage can be adjusted upwards or downwards to achieve desired levels in the subject. Therapeutically effective amounts can also be determined from animal models. The applied dose can be adjusted based on the relative bioavailability and potency of the administered composition. Adjusting the dose to achieve maximal efficacy based on the methods described above and other methods are within the capabilities of those of ordinary skill in the art. These doses can be adjusted using no more than routine experimentation.

In administering the compositions of the invention to a subject, dosing amounts, dosing schedules, routes of administration, and the like may be selected so as to affect known activities of these compositions. Dosages may be estimated based on the results of experimental models, optionally in combination with the results of assays of compositions of the present invention. Dosage may be adjusted appropriately to achieve desired compositional levels, local or systemic, depending upon the mode of administration. The doses may be given in one or several administrations per day. In the event that the response of a particular subject is insufficient at such doses, even higher doses (or effectively higher doses by a different, more localized delivery route) may be employed to the extent that subject tolerance permits. Multiple doses per day are also contemplated in some cases to achieve appropriate systemic levels of the composition within the subject or within the active site of the subject.

The dose of the composition to the subject may be such that a therapeutically effective amount of the composition reaches the active site of the composition within the subject, i.e., optic nerve and/or spinal cord. The dosage may be given in some cases at the maximum amount while avoiding or minimizing any potentially detrimental side effects within the subject. The dosage of the composition that is actually administered is dependent upon factors such as the final concentration desired at the active site, the method of administration to the subject, the efficacy of the composition, the longevity of the composition within the subject, the timing of administration, the effect of concurrent treatments (e.g., as in a cocktail), etc. The dose delivered may also depend on conditions associated with the subject, and can vary from subject to subject in some cases. For example, the age, sex, weight, size, environment, physical conditions, or current state of health of the subject may also influence the dose required and/or the concentration of the composition at the active site. Variations in dosing may occur between different individuals or even within the same individual on different days. It may be preferred that a maximum dose be used, that is, the highest safe dose according to sound medical judgment. Preferably, the dosage form is such that it does not substantially deleteriously affect the subject. In certain embodiments, the composition may be administered to a subject as a preventive measure. In some embodiments, the inventive composition may be administered to a subject based on demographics or epidemiological studies, or to a subject in a particular field or career.

### Methods of Treatment

Provided herein are methods for treating NMO in a subject and/or immunizing a subject against NMO.

Thus in one aspect, the invention provides a method for treating an individual having neuromyelitis optica (NMO), comprising administering a therapeutically effective dose of loop C peptide of aquaporin-4 (AQP4) water channel, or a therapeutically effective fragment or variant thereof. In certain aspects, the composition can be further administered with an immunosuppression therapy. The immunosuppression therapy can be selected from the group consisting of a glucocorticoid agent, a cytostatic agent, an antibody, an immunophilin-acting agent, an interferon, an opioid, a TNF binding protein, and a mycophenolate. The immunosuppression therapy can also be an alkylating agent, nitrogen mustard (cyclophosphamide), nitrosoureas, platinum compound, methotrexate, azathioprine, mercaptopurine, fluorouracil, protein synthesis inhibitor, dactinomycin, antracyclines, mitomycin C, bleomycin, mithramycin, IL-2 receptor-directed antibody, CD3-directed antibody, muromonab-CD3, ciclosporin (Sandimmune®), tacrolimus (Prograf®), sirolimus (Rapamune®), IFN-beta, infliximab (Remicade®), etanercept (Enbrel®), adalimumab (Humira®), fingolimod, leflunomide, rituximab (Rituxan®, MabThera®, or Zytux®), eculizumab (Soliris®), interferon beta-1a (Avonex®), natalizumab (Tysabri®), and malononitriloamides (MNA).

Thus in one aspect, the invention provides a method for treating an individual having neuromyelitis optica (NMO), comprising administering a therapeutically effective dose of loop C peptide of aquaporin-4 (AQP4) water channel, or an therapeutically effective fragment or variant thereof. In certain aspects, the composition can be further administered with an immunosuppression therapy. The immunosuppression therapy can be selected from the group consisting of a glucocorticoid agent, a cytostatic agent, an antibody, an immunophilin-acting agent, an interferon, an opioid, a TNF binding protein, and a mycophenolate. The immunosuppression therapy can also be an alkylating agent, nitrogen mustard (cyclophosphamide), nitrosoureas, platinum compound, methotrexate, azathioprine, mercaptopurine, fluorouracil, protein synthesis inhibitor, dactinomycin, antracyclines, mitomycin C, bleomycin, mithramycin, IL-2 receptor-directed antibody, CD3-directed antibody, muromonab-CD3, ciclosporin (Sandimmune®), tacrolimus (Prograf®), sirolimus (Rapamune®), IFN-beta, infliximab (Remicade®), etanercept (Enbrel®), adalimumab (Humira®), fingolimod, leflunomide, rituximab (Rituxan®, MabThera®, or Zytux®), eculizumab (Soliris®), interferon beta-1a (Avonex®), natalizumab (Tysabri®), and malononitriloamides (MNA).

For treatment, the amount administered is an amount effective in producing the desired effect. An effective amount can be provided in one or a series of administrations. An effective amount can be provided in a bolus or by continuous perfusion.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the antigen-binding fragment administered.

### Kits

In certain aspects, the invention provides a pharmaceutical kit for treating an individual having neuromyelitis optica (NMO), comprising a therapeutically effective dose of loop C peptide of aquaporin-4 (AQP4) water channel, or an therapeutically effective fragment or variant thereof, and instructions for treating said individual. In some embodiments, the kit comprises a sterile container which contains a therapeutic agent of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

Optionally, the kit is a diagnostic kit, e.g., a kit comprising a peptide sequence of loop C AQP4 polypeptide (e.g., SEQ ID NO: 8) and instructions for its use, to identify a subject in which T cell activation occurs upon contact with the peptide sequence of loop C AQP4 polypeptide, thereby identifying a subject having NMO.

If desired the kit is provided together with instructions for administering the compositions to a subject having or at risk of developing NMO. The instructions will generally include information about the use of the composition for the treatment or prevention of NMO. In other embodiments, the instructions include at least one of the following: description of the therapeutic agent; dosage schedule and administration for treatment or prevention of NMO or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

Recombinant methods are well known in the art. The practice of the invention employs, unless otherwise indicated, conventional techniques of molecular biology (including recombinant techniques), microbiology, cell biology, biochemistry and immunology, which are within the skill of the art. Such techniques are explained fully in the literature, such as, “Molecular Cloning: A Laboratory Manual”, second edition (Sambrook et al., 1989); “Oligonucleotide Synthesis” (Gait, ed., 1984); “Animal Cell Culture” (Freshney, ed., 1987); “Methods in Enzymology” (Academic Press, Inc.); “Handbook of Experimental Immunology” (Wei & Blackwell, eds.); “Gene Transfer Vectors for Mammalian Cells” (Miller & Calos, eds., 1987); “Current Protocols in Molecular Biology” (Ausubel et al., eds., 1987); “PCR: The Polymerase Chain Reaction”, (Mullis et al., eds., 1994); and “Current Protocols in Immunology” (Coligan et al., eds., 1991). These techniques are applicable to the production of the polynucleotides and polypeptides, and, as such, can be considered in making and practicing the invention. Particularly useful techniques for are discussed in the sections that follow.

This invention is further illustrated by the following examples which should not be construed as limiting. The contents of all references and published patents and patent applications cited throughout the application are hereby incorporated by reference.

### EXAMPLES

This invention is further illustrated by the following examples which should not be construed as limiting. The contents of all references, GenBank Accession and Gene numbers, and published patents and patent applications cited throughout the application are hereby incorporated by reference. Those skilled in the art will recognize that the invention may be practiced with variations on the disclosed structures, materials, compositions and methods, and such variations are regarded as within the ambit of the invention.

### Example 1

### Neuromyelitis Optic (NMO) Animal Model Generated by AQP4-Reative T Cells Identified AQP4-Reactive T Cells as a Treatment Target for NMO Using an Antigen-Specific Therapy Based on Soluble Loop C Peptide

A unique approach was employed to raise pathogenic AQP4-reactive T cells in AQP4 null mice, which caused an NMO-like disease when adoptively transferred to wild-type mice. Polarization of AQP4-reactive T cells to the T helperl7 phenotype enhanced the phenotype and led to inflammation and demyelination in the optic nerves and spinal cord. In particular, a seronegative model of NMO using pathogenic AQP4-reactive T cells in mice was generated by immunizing AQP4 null mice with peptides corresponding to the second extracellular loop of AQP4, loop C (e.g., loop C sequence-containing peptide SEQ ID NO: 8), which when polarized to a Th17 phenotype and transferred to wild-type mice caused tail and limb weakness. Histology showed demyelination and T cell infiltration throughout the spinal cord and optic nerve, as well as the brain. It was surprisingly identified that AQP4-reactive T cells were sufficient to trigger an NMO-like disease in mice without anti-AQP4 antibodies, indicating that pathogenic AQP4-reactive T cells likely perform a similar role in humans. There was no other evidence of solid organ inflammation despite widespread AQP4 expression in the mouse, supporting the specificity of this approach to modeling the human NMO disease.

Materials and methods

### Animals

Aquaporin-4 null mice backcrossed onto the C57BL/6 background were obtained from Erlend Nagelhus (University of Oslo, Oslo, Norway) and bred in-house. Female C57BL/6 mice between 6-8 weeks of age were purchased from The Jackson Laboratory. All mice were housed in a pathogen-free 12 hour artificial lightdark cycle and had ad libitum access to food and water. The Johns Hopkins Institutional Animal Care and Use Committee approved all experimental procedures.

### Samples

Human IgG fractions were purified from the plasma of patients undergoing plasma exchange using a resin based purification method (Melon Gel IgG Purification kit, Thermo Scientific) two days prior to injection. The purified IgG was concentrated by spin column centrifugation (Amicon Ultra,100 kD MW cut off) and the final protein concentration was adjusted to 25 mg/ml for 100 μl intraperitoneal injection. All NMO patients tested seropositive for the NMO-IgG by the Mayo clinical NMO-IgG assay and the NMO plasma samples from 3 patients were pooled prior to purification. Human control IgG fraction (controllgG) was obtained. All samples were obtained through a protocol approved by the Johns Hopkins Institutional Review Board in a deidentified manner with informed consent to use the samples for research.

### T Cell Generation and Culturing

Aquaporin-4 extracellular loop peptides (human 56-69, 135-53, and 212-30) were synthesized at the Johns Hopkins Synthesis & Sequencing Core Facility. Stock solutions of 120 mg/ml were prepared in DMSO. All three peptides were further diluted into phosphate-buffered saline each at 2 mg/ml and mixed 1:1 with complete adjuvant containing 8 mg/ml heat-killed M. tuberculosis H37Ra (Difco) in incomplete adjuvant (Imject; Thermo-Fisher) (14). Aquaporin-4 KO and syngeneic C57B1/6 mice (Jackson, Mass., USA) were immunized in the flanks with a total of 1000 of emulsion. Mice were also injected intraperitoneally with 250 ng of Pertussis toxin (Tocris) on days 0 and 2. Twenty three days following immunization, spleens were harvested and singe cell suspensions were prepared by pushing spleens through 70 μm cell strainers (Becton Dickenson) using syringe plungers. Red blood cells were depleted by resuspending each spleen in 2 ml of ACK lysis solution (Quality Biological, MD, USA) for 2 minutes at room temperature, followed by washing with media. Cells were counted and seeded in 96-well flat bottom plates at 3×105 cells per well in RPMI 1640 supplemented with Glutamax, 1% non-essential amino acids, 1% sodium pyruvate, 1% antibiotic-antimycotic (Life Technologies, Inc.), 10% fetal calf serum (Sigma) and 50 μM betamercaptoethanol (Sigma). One hundred microliters of media containing peptide (final concentration: 10 n/ml): MOG₃₅₋₅₅, Aqp4₅₆₋₆₉ (loop A), Aqp⁴₁₃₅₋₅₃ (loop C-related), or Aqp4₂₁₂₋₃₀ (loop E) were added to wells in triplicate. Media with no peptide added or containing 0.1% DMSO served as “no stimulus (NS)” background controls. After 4 days in culture in an incubator at 37° C. with 5% CO₂ in a humidified atmosphere, 10 μl of a solution containing 0.5 μCu of ³H-thymidine (Perkin-elmer) were added to each well and incubated a further 18 hours. Cells were harvested onto filter paper mat. After drying, mats were treated with scintillation fluid and assayed for ³H incorporation. Results are expressed as counts per minute (cpm).

### T Cell Generation, Adoptive Transfer and Behavioral Scoring

In initial experiments, immunization of AQP4 null mice with peptide corresponding to the second extracellular loop of AQP4, loop C, (SEQ ID NO: 6) in DMSO/PBS was injected with Freund's adjuvant subcutaneously on day 0. On day 23, spleens were harvested, bulk NH4-lysed and cultured in standard media. For T cell proliferation assays, peptides at a final concentration of 10 mg incomplete Freund's adjuvant containing 12.5 mg/ml heatkilled Mycobacterium tuberculosis; each animal therefore received 625 μg M. tuberculosis (day 0). Pertussis Toxin (300 ng) was administered intraperitoneally on days 0 and 2. Animals were weighed daily and scored on a standardized 5-point disability scale by a blinded examiner (Jones et al. 2008). A series of 4 intraperitoneal injections of human IgG purified from either pooled NMO plasma or control human plasma were administered on days 13, 14, 18, and 19 for a total of 10 mg/animal. Vehicle controls received an equal volume of PBS.

In further experiments, six to seven week old female aquaporin-4 KO mice were immunized with an emulsion of 4 mg/ml Complete Freund's adjuvant with 1 mg/ml of Loop C-related peptide (135-53) on each flank and each shoulder (50 μl per injection site). Ten days following immunization, lymph nodes (inguinal, lumbar, brachial and axillary) were collected. Single cell suspensions were prepared by forcing nodes through a 70 μm cell strainer using a syringe plunger. After washing in complete media (RPMI1640 with 10% FCS, 50 μM β-mercaptoethanol, non-essential amino acids, sodium pyruvate, HEPES), cells were counted and seeded into T75 flasks at 6×10⁶ cells per ml. Unpolarized cells were stimulated with peptide only (final concentration was 50 μg/ml). For Th17 polarization, cells were stimulated with 30 ng/ml IL-6, 20 ng/ml IL-23 (eBiosciences) and 10 μg/ml of anti-IFNgamma (XMG1.2; BD Biosciences) in addition to peptide. Following 3 days of culture, cells were harvested, washed with sterile PBS, and 5×10⁶ cells were injected into mice intravenously through their tail veins. Pertussis toxin (250 ng; Tocris) was injected intravenously immediately following cells and again two days later. Behavioral signs and weights were tracked starting 5 days post-transfer of cells, which was quantified using a standardized 5-point EAE disability scale by a blinded examiner (14). Animals were euthanized and tissues harvested for histological evaluation 14-21 days following cell transfer.

### ELISPOT Assay

ELISPOT assay was used to determine the frequency of cytokine-producing cells in polarized and unpolarized cell cultures. The day before cells were to be harvested from immunized AQP4 null mice, the wells of an immobilon P-bottom 96-well plates (Multiscreen®HTS, 0.45 μm pore size; EMB Millipore, USA) were pre-wet with 35% ethanol for 30 seconds, washed three times with coating buffer, and coated with 50 μl of a 1:250 dilution of capture antibody, anti-IL17 (Th17), provided with ELISPOT Ready-Set-Go kits (eBioscience). Plates were covered and incubated overnight at 4° C. Wells were then washed twice with coating buffer and once with complete media; plates were stored in the incubator until cells were prepared. Spleen and lymph node cells were prepared as described above and prepared with and without polarizing conditions. Harvested cells were 2-fold serially diluted in media containing 3×10⁷ irradiated splenocytes (irradiated with 3,500 rads) as antigen-presenting cells (APCs); APCs mixed 12:1 with 1.25×10⁵ lymph node cells per well yielded the most well-defined spots. After overnight in culture, wells were washed (TBS+0.05% Tween®-20). Detection antibody was diluted 1:250 in diluents provided in the kit and 50 μl applied to each well for 2 hours at RT. After washing, streptavidin-alkaline phosphatase was added to each well (1:2500; Sigma) for 45 minutes. After further washing, signal was developed with development solution containing BCIP and NBT (i.e. 150 mM Tris-HCl, 5 mM MgCl2, 100 mM NaCl, pH9.5 supplemented with 4 mM levamisole (Sigma), 0.15 mg/ml 5-bromo-4-chloro-3-indolyl-phosphate, and 0.36 mg/ml 4-nitro blue tetrazolium chloride (Roche) for 10 minutes at room temperature in the dark. Reaction was stopped by washing with PBS then distilled H₂O before air drying. Spots were imaged with an Immunospot Series 3 Analyzer (Cellular Technology, Ltd.) and counted using Image J and the “find Maxima” function. Results are expressed as the mean of triplicate values (±standard error of the mean, SEM) adjusted to per 10⁶ cells per well. Student's t-test was performed on data; p<0.05 was considered statistically significant.

### Tissue Processing and Histology

Animals were anesthetized with isofluorane and perfused via cardiac puncture first with PBS and then with freshly prepared 4% paraformaldehyde solution. The optic nerves and spinal cords were harvested, fixed overnight, cryopreserved in 30% sucrose and frozen for sectioning. After embedding tissue in in O.C.T. Compound (TissueTek®), ten to thirty micron slices sections were mounted on Superfrost Plus Microscope Slides (Fisher brand). The first cohort of animals was sacrificed 20 minutes after the last intraperitoneal injection of human IgG for the purpose of tracking human antibody entrance into the mouse central nervous system. The second cohort of animals was sacrificed and their tissue was prepared in a similar fashion on day 62 post-disease induction.

### Eriochrome Cyanine Staining for Myelin & Immunihistochemical Staining

Eriochrome cyanine was used to identify demyelinating lesions in the sectioned tissue. Eriochrome cyanine solution was prepared by dissolving eriochrome cyanine in 450 ml 0.5% H₂SO₄ (0.2%) and 10% FeCl₃ added to a final concentration of 0.4%. The sectioned tissue was hydrated by serial washes in 100% ethanol, 95% ethanol, 70% ethanol and distilled water for 10 minutes each and then immersed for 15 minutes in eriochrome cyanine solution. After staining, differentiation was carried out in freshly made 0.1% NH₄OH for 20-30 seconds and halted by thorough washing in distilled water. Slides were mounted as described below. Sections were counterstained with 0.1% eosin Y in acetate buffer. Immunohistochemical staining for CD3+ T cells was performed by washing sections in saline before performing heat-mediated antigen retrieval in 0.05M sodium borate buffer (pH 8.0) in a microwave pressure cooker. Slides immersed in buffer were heated in the microwave at full power until full pressure was achieved (5 minutes) then heated for an additional 7 minutes at 20% power. After 3 minutes of cooling and flooding of slide container with room temperature saline, slides were transferred to 3% H₂O₂ for 20 minutes to quench endogenous peroxidases and blocked for endogenous biotin using an avidin/biotin blocking kit (Vector Laboratories, Inc.). Non-specific binding was blocked with 5% goat serum in 0.1% Triton®X-100 for 30 minutes at room temperature. Anti-CD3 rabbit monoclonal antibody (clone SP7; Gene Tex,USA) was applied at 1:75 overnight at 4° C. and detected with biotinylated goat anti-rabbit IgG (1:1000; Vector Laboratories, Inc.), followed by Avidin-Biotin Complex-horse radish peroxidase (Vector Laboratories, Inc.). Signal was developed with 0.5 mg/ml diaminobenzidine HCl in PBS with 0.03% H₂O₂ for 5 minutes. After washing, slides were Fast Green counterstained, dehydrated and mounted. Quantification and analysis of myelin and CD3 staining was as described (7). Glial fibrillary acidic protein (GFAP), myelin basic protein for myelin, and aquaporin-4 were examined by immunofluorescence (without antigen retrieval) applying mouse anti-GFAP (1:1000; Sigma), rabbit monoclonal anti-MBP (1:250; Epitomics/Abcam), and rabbit anti-AQP4 (H-19) (1:250; Santa Cruz Biotechnology) overnight at 4° C., followed by goat Alexa Fluor® 555-conjugated anti-rabbit IgG and Alexa Fluor® 488-conjugated anti-mouse IgG (1:250; Life Technologies/Molecular Probes) for 30 minutes at RT. Fluorescent sections were mounted with Fluorogel (Electron Microscopy Sciences) containing 2 μg/ml 4′,6-Diamidino-2-Phenylindole (DAPI) and sealed with clear nail polish.

In the current example, it was initially identified that wildtype C57B1/6 mice did not develop encephalomyelitis after immunization with peptides of AQP4, despite generating T cell responses against several immunodominant peptides. As the human NMO anti-AQP4 antibody targets an extracellular epitope of AQP4, antibody production and T cell responses against peptides corresponding to the extracellular loops of AQP4 loops A, C and E were screened for in AQP4 null mice. No evidence of antibody production against any of the loops was identified; however, there was a robust T cell response against the second extracellular loop of AQP4, loop C (data not shown).

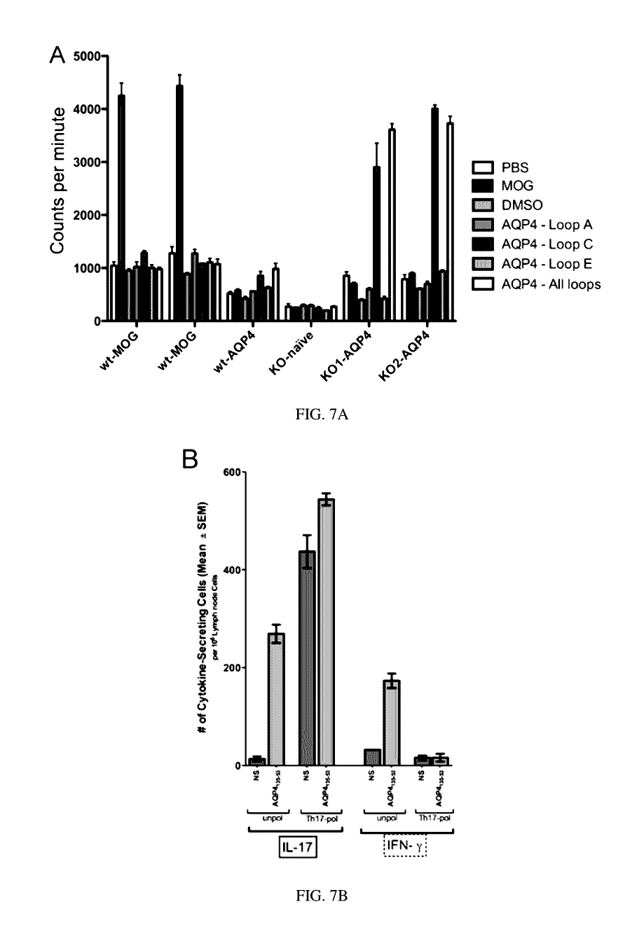

In NMO, the anti-AQP4 antibody targets an extracellular epitope of AQP4. Using AQP4 null mice, antibody production and T cell responses against peptides corresponding to the extracellular loops of AQP4, loops A, C (specifically to loop C-related peptide sequence 135-153 of SEQ ID NO: 8) and E was examined. AQP4 null mice were used in this model because these mice did not have to overcome immune tolerance to develop pathogenic AQP4-reactive T cells. Although no evidence of antibody production against any of the loops was identified, there was a robust T cell response against the loop C-related peptide sequence 135-153 of SEQ ID NO: 8 (FIG. 7A). The lack of anti-AQP4 antibody production was not unexpected, as short peptides do not routinely exhibit robust antibody responses. Unpolarized AQP4-reactive T cells produced a significant number of interleukin-17 (IL17) and IFN-gamma secreting cells, compared to non-stimulus controls (FIG. 7B). However, when polarized solely to the Th17 phenotype, the number of IL17-secreting cells were doubled and the number of IFN-gamma producing cells were barely detectable (FIG. 7B).

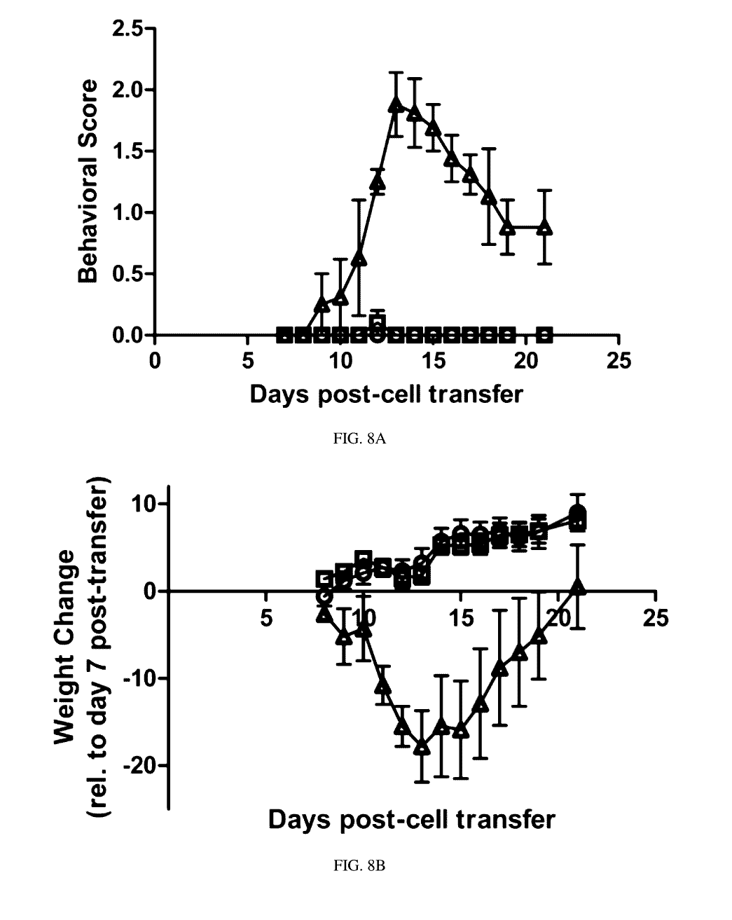

AQP4 null mice that generated a robust T cell reaction against the loop C-related peptide sequence 135-153 of SEQ ID NO: 8, did not develop autoimmune neurological disease, as they did not express the target antigen. Even when intravenously transferred to wildtype AQP4-expressing C57B1/6 mice, these AQP4-reactive T cells did not develop clinically meaningful behavioral manifestations or histological evidence of CNS demyelinating disease beyond meningeal inflammation (data not shown). Prior to adoptive transfer, when AQP4-reactive T cells were polarized to a stronger pro-inflammatory Th17 helper cell phenotype, the clinical effect was dramatic with leg weakness and paralysis in addition to a drooping tail with behavioral scores of at least 2.0 (FIG. 8A) and associated weight loss (FIG. 8B).

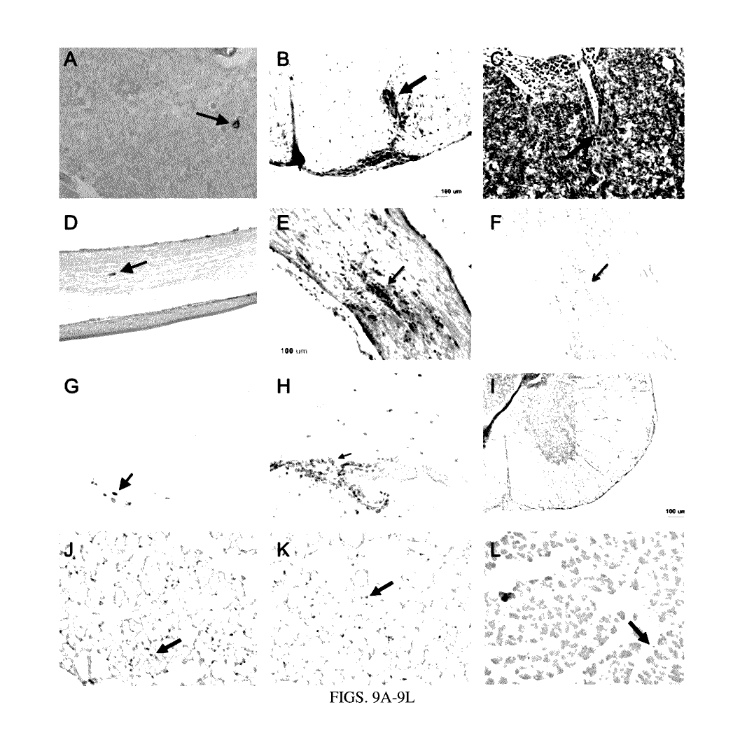

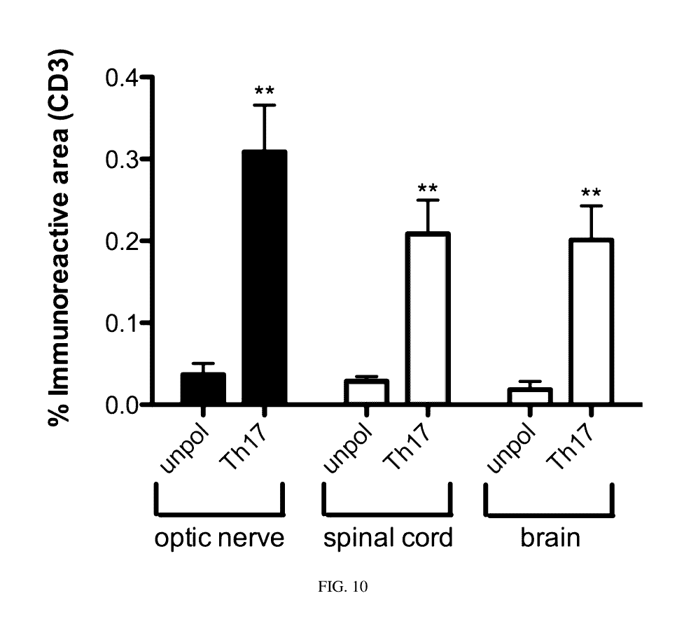

Three important controls showed no clinical or histological phenotype, confirming the specificity of this model. First, adoptive transfer of Th17polarized AQP4-reactive T cells from unstimulated cultures or cultures stimulated with non-CNS proteins was harmless, which highlighted that AQP4-peptide was required during the polarization process (FIGS. 8A-8B). Second, AQP4-reactive T cells polarized to Th17 that were transferred back to naïve AQP4 null mice were also harmless, demonstrating that astrocytic expression of AQP4 in the host mouse was necessary for this model (data not shown). Histologically, in clinically asymptomatic wildtype recipients of unpolarized AQP4-reactive T cells, there were rare CD3+ T cells scattered in the parenchyma of the spinal cord, optic nerve and brain (FIGS. 9A, 9D, 9G), as well as other AQP4-expressing solid organs such as the lung (FIG. 9J). In clinically-affected wildtype recipients of adoptively transferred Th17-polarized AQP4-reactive T cells, histology revealed demyelination and increased inflammatory infiltrate comprised primarily of CD4+ lymphocytes in the spinal cord, optic nerve and brain. Inflammation and demyelination in the spinal cord (FIGS. 9B, 9C) and optic nerves (FIGS. 9E, 9F) accounted for the majority of symptoms, but parts of the brainstem, cerebellum and cerebral cortex showed areas of inflammation that were not as clinically obvious in the mice (FIG. 9H). Although the AQP4 water channel was targeted by these pathogenic T cells, astrocytic AQP4 expression appeared relatively intact even within acute inflammatory lesions (FIG. 9I). Despite AQP4 expression in many other solid organs, there was no evidence of inflammation or AQP4 loss outside of the CNS including the lung (FIG. 9K) or muscles (FIG. 9L) in clinically affected mice. Blinded quantification of CD3 cells in the spinal cord, optic nerve, and brain of wildtype recipients of Th17-polarized AQP4-reactive T cells showed >5-fold more immunoreactivity (**p<0.01) compared to mice that received unpolarized AQP4-reactive T cells (FIG. 10).

Thus, the above studies demonstrated that a disease similar to NMO could be caused in wild-type mice by adoptive transfer of autoreactive T cells against the second extracellular loop of AQP4, loop C. The above studies also demonstrated that adoptive intravenous transfer of pathogenic AQP4-reactive T cells was sufficient to cause an NMO-like inflammatory disease that attacks the optic nerves and spinal cord while sparing other AQP4-expres sing organs. Polarization of AQP4-reactive T cells to the Th17 phenotype prior to transfer amplified the inflammation, leading to more severe demyelination and neurological dysfunction. The best explanation for this response was that immunization with AQP4-loop C sequence-containing peptide SEQ ID NO: 8 in AQP4 null mice generated a T cell response that was different from immunization of the same loop in wild-type mice (15). The AQP4-reactive T cells in null mice were not exposed to any degree of negative selection, as they likely would have been if raised in wild-type mice. There likely was also a protective regulatory response in wild-type mice that suppressed any potential auto-reactive tendency.

In a previous study using a slightly shorter peptide spanning loop C, immunization elicited a T cell response in C57B1/6 mice, but did not trigger clinical disease of any type. Similarly, immunization with loop C peptide in AQP4 null mice also stimulated a T cell response with no clinical phenotype. The latter was expected, as there was no AQP4 in null mice for AQP4-reactive T cells to attack. However, when the AQP4-reactive T cells from AQP4 null mice were adoptively transferred to wild-type mice, the autoreactive T cells produced an NMO-like disease with CNS inflammation. Polarization of those T cells to the Th17 phenotype, and not the Th1 phenotype, prior to transfer amplified the inflammation, which remained confined to the optic nerves and spinal cord and spares the brain as well as other AQP4-expressing organ in the mouse.

The best explanation for this response was that immunization with AQP4-loop C peptide in AQP4 null mice generates a T cell response that is different from immunization of the same loop in wildtype mice. These AQP4-reactive T cells in null mice are not exposed to any degree of negative selection as they likely would be if raised in wildtype mice. There may also be a protective regulatory response in wildtype mice that suppresses any potential autoreactive tendency.

The implications of this study point to a central immunopathogenic role of TH17 polarized AQP4-reactive T cells of NMO in both triggering and localizing inflammation only to the optic nerves and spinal cord. Pathogenic T cells targeting AQP4 did not kill AQP4-expressing astrocytes and did not cause loss of AQP4 expression. Rather, their role as demonstrated in this model was to trigger attacks directed towards AQP4-rich areas of the CNS and then recruited other components of the immune system, including antibodies and complement, to mediate the astrocytic damage. In human NMO pathology, death of astrocytes and loss of aquaporin-4 was likely initiated with binding of anti-AQP4 antibody, which led to either complement-mediated destruction of the M23 isoform or internalization of the M1 isoform of AQP4 (16, 17). A pathogenic function of the anti-AQP4 antibody in exacerbating neuroinflammation was previously demonstrated, but only in the setting of ongoing experimental myelitis, not in instigating the disease (5-7). Passive transfer of anti-AQP4 antibodies to rats or mice could not trigger neuroinflammation in the absence of autoreactive T cells, even in P2 pups in which the blood brain barrier was underdeveloped and allowed the antibody to bind to CNS targets. Thus, the specificity of the immune target to the nervous system was not mediated by the anti-AQP4 antibody, and the anti-AQP4 antibody did not discriminate among AQP4 targets, as it could bind in any organ.

The extracellular loop C is the most common target of the anti-AQP4 antibody in humans (19). AQP4-reactive T cells and a pathogenic anti-AQP4 antibody likely work together to cause NMO. In this model, a susceptible person is exposed to a peptide corresponding to loop C of AQP4 under conditions that stimulate both an autoreactive T cell and antibody response. A Th17 response to AQP4 likely causes a more fulminant disease, as was demonstrated in this study and previous animal models (20). Once AQP4-reactive T cells trigger inflammation directed to the optic nerves and spinal cord, anti-AQP4 exacerbates the pathology by fueling complement activation and granulocyte recruitment.

This model highlights the potential for AQP4-specific immunotherapy for NMO. As a disease with a highly specific antigen (AQP4) and antibody response (antiAQP4), and now likely associated with AQP4-reactive T cells, NMO is poised for treatment with an antigen-specific therapy (21, 22). To induce a tolerance response, high dose soluble loop C peptide (e.g., loop C sequence-containing peptide SEQ ID NO: 8 and/or fragments or derivatives thereof) can be provided to patients in the setting of immunosuppression with a drug like rituximab, commonly used to treat NMO currently. With pre-existing disease, an oral route to achieve mucosal tolerance is likely to be the safest initial approach to avoid worsening the disease (23). In other diseases with less antigen-specificity (where antigen-specific therapy is being tested), such as rheumatoid arthritis and multiple sclerosis, there is significant heterogeneity in the immunodominant antigen responses; in contrast, NMO is largely an AQP4-related disease, defined by reaction solely to the AQP4 water channel although the precise target within AQP4 likely varies slightly among NMO patients, with some patients producing antibody responses against loops A and E as well (19). Without wishing to be bound by theory, it appears that the most pathogenic antibodies target loop C (e.g., loop C sequence-containing peptide SEQ ID NO: 8 and/or fragments or derivatives thereof) of AQP4, but some patients produce antibody responses against loops A and E as well (19). A study in Lewis rats in which T cells reactive against loop E could induce inflammation in the spinal cord suggested that extracellular targets of AQP4 other than loop C are likely involved as well (24).

### Equivalents:

Those skilled in the art will recognize, or be able to ascertain using no more than routine experimentation, many equivalents to the specific embodiments and methods described herein. Such equivalents are intended to be encompassed by the scope of the following claims.

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

### REFERENCES

1. Oh J, Levy M. 2012. Neuromyelitis optica: an antibody-mediated disorder of the central nervous system. Neurol Res Int 2012: 460825.
2. Matsuoka T, Suzuki S O, Suenaga T, Iwaki T, Kira J. 2011. Reappraisal of aquaporin-4 astrocytopathy in Asian neuromyelitis optica and multiple sclerosis patients. Brain Pathol 21: 516-32.
3. Popescu B F, Lucchinetti C F. 2012. Pathology of demyelinating diseases. Annu Rev Pathol 7: 185-217.
4. Bennett J L, Lam C, Kalluri S R, Saikali P, Bautista K, Dupree C, Glogowska M, Case D, Antel J P, Owens G P, Gilden D, Nessler S, Stadelmann C, Hemmer B. 2009. Intrathecal pathogenic anti-aquaporin-4 antibodies in early neuromyelitis optica. Ann Neurol 66: 617-29.
5. Bradl M, Misu T, Takahashi T, Watanabe M, Mader S, Reindl M, Adzemovic M, Bauer J, Berger T, Fujihara K, Itoyama Y, Lassmann H. 2009. Neuromyelitis optica: pathogenicity of patient immunoglobulin in vivo. Ann Neurol 66: 630-43.
6. Kinoshita M, Nakatsuji Y, Kimura T, Moriya M, Takata K, Okuno T, Kumanogoh A, Kajiyama K, Yoshikawa H, Sakoda S. 2009. Neuromyelitis optica: Passive transfer to rats by human immunoglobulin. Biochem Biophys Res Commun 386: 623-7.
7. Saini H, Rifkin R, Gorelik M, Huang H, Ferguson Z, Jones MV, Levy M. 2013. Passively transferred human NMO-IgG exacerbates demyelination in mouse experimental autoimmune encephalomyelitis. BMC Neurol 13: 104.
8. Ratelade J, Asavapanumas N, Ritchie A M, Wemlinger S, Bennett J L, Verkman A S. 2013. Involvement of antibody-dependent cell-mediated cytotoxicity in inflammatory demyelination in a mouse model of neuromyelitis optica. Acta Neuropathol 126: 699-709.
9. Kinoshita M, Nakatsuji Y, Kimura T, Moriya M, Takata K, Okuno T, Kumanogoh A, Kajiyama K, Yoshikawa H, Sakoda S. 2010. Anti-aquaporin-4 antibody induces astrocytic cytotoxicity in the absence of CNS antigen-specific T cells. Biochem Biophys Res Commun 394: 205-10.
10. Saadoun S, Waters P, Bell B A, Vincent A, Verkman A S, Papadopoulos M C. 2010. Intra-cerebral injection of neuromyelitis optica immunoglobulin G and human complement produces neuromyelitis optica lesions in mice. Brain 133: 349-61.
11. Saadoun S, Waters P, Macdonald C, Bridges L R, Bell B A, Vincent A, Verkman A S, Papadopoulos M C. 2011. T cell deficiency does not reduce lesions in mice produced by intracerebral injection of NMO-IgG and complement. J Neuroimmunol 235: 27-32.
12. Nelson P A, Khodadoust M, Prodhomme T, Spencer C, Patarroyo J C, Varrin-Doyer M, Ho J D, Stroud R M, Zamvil S S. 2010. Immunodominant T cell determinants of aquaporin-4, the autoantigen associated with neuromyelitis optica. PLoS One 5: e15050.
13. Kalluri S R, Rothhammer V, Staszewski O, Srivastava R, Petermann F, Prinz M, Hemmer B, Korn T. 2011. Functional characterization of aquaporin-4 specific T cells: towards a model for neuromyelitis optica. PLoS One 6: e16083.
14. Jones M V, Nguyen T T, Deboy C A, Griffin J W, Whartenby K A, Kerr D A, Calabresi P A. 2008. Behavioral and pathological outcomes in MOG 35-55 experimental autoimmune encephalomyelitis. J Neuroimmunol 199: 83-93.
15. Lin R H, Stockinger B. 1989. T cell immunity or tolerance as a consequence of self antigen presentation. Eur J Immunol 19: 105-10.
16. Hinson S R, Romero M F, Popescu B F, Lucchinetti C F, Fryer J P, Wolburg H, Fallier-Becker P, Noell S, Lennon V A. 2012. Molecular outcomes of neuromyelitis optica (NMO)-IgG binding to aquaporin-4 in astrocytes. Proc Natl Acad Sci USA 109: 1245-50.
17. Rossi A, Ratelade J, Papadopoulos M C, Bennett J L, Verkman A S. 2012. Neuromyelitis optica IgG does not alter aquaporin-4 water permeability, plasma membrane M1/M23 isoform content, or supramolecular assembly. Glia 60: 2027-39.
18. Saadoun S, Waters P, Leite M I, Bennett J L, Vincent A, Papadopoulos M C. 2013. Neuromyelitis optica IgG causes placental inflammation and fetal death. J Immunol 191: 2999-3005.
19. Iorio R, Fryer J P, Hinson S R, Fallier-Becker P, Wolburg H, Pittock S J, Lennon V A. 2013. Astrocytic autoantibody of neuromyelitis optica (NMO-IgG) binds to aquaporin-4 extracellular loops, monomers, tetramers and high order arrays. J Autoimmun 40: 21-7.
20. Herges K, de Jong B A, Kolkowitz I, Dunn C, Mandelbaum G, Ko R M, Maini A, Han M H, Killestein J, Polman C, Goodyear A L, Dunn J, Steinman L, Axtell R C. 2012. Protective effect of an elastase inhibitor in a neuromyelitis optica-like disease driven by a peptide of myelin oligodendroglial glycoprotein. Mult Scler 18: 398-408.
21. Vaknin-Dembinsky A, Brill L, Kassis I, Petrou P, Ovadia H, Ben-Hur T, Abramsky O, Karussis D. 2012. T-cell reactivity against AQP4 in neuromyelitis optica. Neurology 79: 945-6.
22. Varrin-Doyer M, Spencer C M, Schulze-Topphoff U, Nelson P A, Stroud R M, Cree B A, Zamvil S S. 2012. Aquaporin 4-specific T cells in neuromyelitis optica exhibit a Th17 bias and recognize Clostridium ABC transporter. Ann Neurol 72: 53-64.
23. Harrison L C. 2008. Vaccination against self to prevent autoimmune disease: the type 1 diabetes model. Immunol Cell Biol 86: 139-45.
24. Pohl M, Fischer M T, Mader S, Schanda K, Kitic M, Sharma R, Wimmer I, Misu T, Fujihara K, Reindl M, Lassmann H, Bradl M. 2011. Pathogenic T cell responses against aquaporin 4. Acta Neuropathol 122: 21-34.

1. A pharmaceutical composition for treating neuromyelitis optica (NMO) comprising a therapeutically effective amount of loop C peptide of aquaporin-4 (AQP4) water channel, or a therapeutically effective fragment or variant thereof.

2. The pharmaceutical composition of claim 1, wherein the neuromyelitis optica (NMO) is monophasic neuromyelitis optica (NMO).

3. The pharmaceutical composition of claim 1, wherein the neuromyelitis optica (NMO) is relapsing neuromyelitis optica (NMO).

4. The pharmaceutical composition of claim 1, wherein loop C peptide of aquaporin-4 (AQP4) water channel is SEQ ID NO: 1 or a polypeptide having at least 90% sequence identity therewith.

5. The pharmaceutical composition of claim 1, wherein the therapeutically effective amount is sufficient to induce a tolerance response.

6. The pharmaceutical composition of claim 1, wherein the composition further comprises an immunosuppression therapy.

7. The pharmaceutical composition of claim 6, wherein the immunosuppression therapy is selected from the group consisting of a glucocorticoid agent, a cytostatic agent, an antibody, an immunophilin-acting agent, an interferon, an opioid, a TNF binding protein, and a mycophenolate.

8. The pharmaceutical composition of claim 6, wherein the immunosuppression therapy is an alkylating agent, nitrogen mustard (cyclophosphamide), nitrosoureas, platinum compound, methotrexate, azathioprine, mercaptopurine, fluorouracil, protein synthesis inhibitor, dactinomycin, antracyclines, mitomycin C, bleomycin, mithramycin, IL-2 receptor-directed antibody, CD3-directed antibody, muromonab-CD3, ciclosporin (Sandimmune®), tacrolimus (Prograf®), sirolimus (Rapamune®), IFN-beta, infliximab (Remicade®), etanercept (Enbrel®), adalimumab (Humira®), fingolimod, leflunomide, rituximab (Rituxan®, MabThera®, or Zytux®), eculizumab (Soliris®), interferon beta-1a (Avonex®), natalizumab (Tysabri®), and malononitriloamides (MNA).

9-24. (canceled)

25. A method for inducing a tolerance response in an individual having neuromyelitis optica (NMO), comprising administering an immunogenically effective amount of loop C peptide of aquaporin-4 (AQP4) water channel, or an immunogenic effective fragment or variant thereof.

26-32. (canceled)

33. A pharmaceutical kit for treating an individual having neuromyelitis optica (NMO), comprising a therapeutically effective dose of loop C peptide of aquaporin-4 (AQP4) water channel, or a therapeutically effective fragment or variant thereof, and instructions for treating said individual, or
A pharmaceutical kit for treating an individual having neuromyelitis optica (NMO), comprising a therapeutically effective dose of a peptide of 50 or fewer amino acids in length that, when aligned with SEQ ID NO: 8, possesses 17 or more amino acid residues of SEQ ID NO: 8, and instructions for treating said individual.

34-39. (canceled)

40. A pharmaceutical composition for treating neuromyelitis optica (NMO) comprising a therapeutically effective amount of a peptide of 50 or fewer amino acids in length that, when aligned with SEQ ID NO: 8, possesses 17 or more amino acid residues of SEQ ID NO: 8, or
An immunization composition for immunizing a subject having neuromyelitis optica (NMO), comprising an immunogenically effective amount of a peptide of 50 or fewer amino acids in length that, when aligned with SEQ ID NO: 8, possesses 17 or more amino acid residues of SEQ ID NO: 8.

41-55. (canceled)

56. A method for treating an individual having neuromyelitis optica (NMO), comprising administering a peptide of 50 or fewer amino acids in length that, when aligned with SEQ ID NO: 8, possesses 17 or more amino acid residues of SEQ ID NO: 8, or
A method for inducing a tolerance response in an individual having neuromyelitis optica (NMO), comprising administering a peptide of 50 or fewer amino acids in length that, when aligned with SEQ ID NO: 8, possesses 17 or more amino acid residues of SEQ ID NO: 8.

57-75.

76. The composition of claim 40, further comprising a therapeutically effective amount of loop A and/or B peptide of aquaporin-4 (AQP4) water channel, or a therapeutically effective fragment or variant thereof.

77. The method of claim 56, further comprising the step of administering a therapeutically effective amount of a second NMO treatment.

78. The method of claim 56, further comprising the step of administering a therapeutically effective amount of loop A and/or B peptide of aquaporin-4 (AQP4) water channel, or a therapeutically effective fragment or variant thereof.

79-80. (canceled)

81. A method for detecting NMO in a subject, the method comprising:
a) obtaining a T cell- and/or antibody-containing sample from a subject;
b) contacting the sample with a peptide consisting of SEQ ID NO: 8 or a fragment or variant thereof in an amount sufficient to allow for formation of a SEQ ID NO: 8-specific antibody-SEQ ID NO: 8 peptide complex or to allow for T cell activation in a SEQ ID NO: 8-specific manner; and
c) detecting T cell activation or formation of a SEQ ID NO: 8-specific antibody-SEQ ID NO: 8 peptide complex, wherein T cell activation or formation of the SEQ ID NO: 8-specific antibody-SEQ ID NO: 8 peptide complex indicates that the subject has NMO,
thereby detecting NMO in the subject, or
A kit for detecting NMO in a subject, the kit comprising a peptide of 50 or fewer amino acids in length that, when aligned with SEQ ID NO: 8, possesses 17 or more amino acid residues of SEQ ID NO: 8 and that, when contacted with a sample of a subject induces T cell activation, thereby indicating NMO in the subject, and instructions for its use, or
An NMO model mouse induced by administration to a mouse of a peptide of 50 or fewer amino acids in length that, when aligned with SEQ ID NO: 8, possesses 17 or more amino acid residues of SEQ ID NO: 8, or
A method for identifying a candidate NMO therapeutic compound, the method comprising administering a test compound to the NMO model mouse of claim 86 and identifying improvement of a neurological symptom of NMO in the NMO model mouse in the presence of the test compound, optionally as compared to an appropriate control, thereby identifying the test compound as a candidate NMO therapeutic.

82-87. (canceled)
